(19) United States
(12) Patent Application Publication
(10) Pub. No.: US 2024/0130877 A1
(43) Pub. Date: Apr. 25, 2024

(54) MULTI-COMPONENT DELIVERY SYSTEMS AND METHODS

(71) Applicant: W. L. Gore & Associates, Inc., Newark, DE (US)

(72) Inventors: Merrill J. Birdno, Flagstaff, AZ (US); Jerald M. Crawley, Flagstaff, AZ (US); Kehinde A. Majolagbe, Flagstaff, AZ (US); Adam H. Power, London (CA); Brandon C. Short, Flagstaff, AZ (US); Derek M. Ward, Flagstaff, AZ (US); Patrick S. Young, Flagstaff, AZ (US)

(21) Appl. No.: 18/278,213

(22) PCT Filed: Feb. 22, 2022
(86) PCT No.: PCT/US2022/017333

§ 371 Date: Aug. 21, 2023

Related U.S. Application Data
(60) Provisional application No. 63/152,144, filed on Feb. 22, 2021.

(51) Int. Cl. A61F 2/954; A61F 2/966

(52) U.S. Cl. CPC A61F 2/954; A61F 2/966

(57) ABSTRACT

A method of deploying a multibranch stent graft at a target site having a main lumen and a first branch lumen is provided. The method includes advancing a catheter including a main body having a first portion and a second portion, the main body defining a first portal being pre-cannulated with a first guide member, partially deploying the first portion of the main body, advancing a first sheath along the first guide member through the first portal, advancing a first articulatable wire through the first sheath, positioning the first articulatable wire into a first branch lumen of the target site, partially deploying the second portion of the main body, fully deploying the first portion and the second portion of the main body, advancing a first side branch body along the first articulatable wire into the first branch lumen, and deploying the first side branch body in the first branch lumen.

### CROSS-REFERENCE TO RELATED APPLICATION

This application is a national phase application of PCT Application No. PCT/US2022/017333, internationally filed on Feb. 22, 2022, which claims the benefit of Provisional Application No. 63/152,144, filed Feb. 22, 2021, which are incorporated herein by reference in their entireties for all purposes.

### FIELD

The present disclosure relates generally to systems and methods for delivering multi-component devices. More specifically, the disclosure relates to systems and methods for delivering endovascular devices that include individual components to a target site.

### BACKGROUND

A variety of branched, anatomical passages may benefit from treatment in the form of an implanted, endoluminal device. One such passage is a vascular passage, such as an artery, with an aneurysm. Aortic disease and trauma such as aneurysms and dissections present a significant risk to a patient. That risk is increased based on the patient's condition. Such conditions or factors can include the patient's age and preexisting and/or related conditions such as cardiopulmonary bypass, cardiac arrest, circulatory arrest. These and other factors may limit the patient's ability to withstand and recover from surgery to repair the aortic disease. This same issue exists in other diseased and damaged tissues in the patients.

With respect to aneurysms, in order to prevent rupturing of an aneurysm, a stent graft may be introduced into a blood vessel percutaneously and deployed to span the aneurysmal sac. Stent grafts include a graft fabric secured to a cylindrical scaffolding or framework of one or more stents. The stent(s) provide rigidity and structure to hold the graft open in a tubular configuration as well as the outward radial force needed to create a seal between the graft and a healthy portion of the vessel wall and provide migration resistance. Blood flowing through the vessel can be channeled through the luminal surface of the stent graft to reduce, if not eliminate, the stress on the vessel wall at the location of the aneurysmal sac. Stent grafts may reduce the risk of rupture of the blood vessel wall at the aneurysmal site and allow blood to flow through the vessel without interruption.

Various endovascular repair procedures such as the exclusion of an aneurysm require a stent graft to be implanted adjacent to a vascular bifurcation. Often the aneurysm extends into the bifurcation requiring the stent graft to be placed into the bifurcation. A bifurcated stent graft is therefore required in these cases. Modular stent grafts, having a separate main body and branch component are often preferred in these procedures due to the ease and accuracy of deployment. See U.S. Patent Application No. 2008/0114446 to Hartley et al. for an example of a modular stent graft having separate main body and branch stent components. In the Hartley et al. publication the main body stent has a fenestration in the side wall that is tailored to engage and secure the side branch stent.

### SUMMARY

An endoprosthesis including a main body is provided with side branch portals for providing fluidic access to side branches of a main lumen when the main body of the endoprosthesis is deployed in the main lumen. A method of deployment of the endoprosthesis is also provided

According to one example (“Example 1”), a method of deploying includes a multibranch stent graft at a target site having a main lumen and a first branch lumen is provided, the method including advancing a main guidewire to a target site; advancing a catheter including a main body of a multibranch stent graft along the main guidewire toward the main lumen of the target site, the main body having a first portion and a second portion, the main body defining a first portal operable to provide fluidic access from the main body to a first side branch extending from the target site when the main body is deployed at the target site, the first portal being pre-cannulated with a first secondary guidewire prior to advancing the main body along the main guidewire; partially deploying the first portion of the main body in the main lumen of the target site; advancing a first sheath along the first guide member through the first portal; advancing a first articulatable wire or guide catheter through the first sheath; positioning the first articulatable wire or guide catheter into a first branch lumen of the target site; partially deploying the second portion of the main body in the main lumen of the target site; fully deploying the first portion and the second portion of the main body; advancing a first side branch body along the first articulatable wire or guide catheter into the first branch lumen of the target site; and deploying the first side branch body in the first branch lumen of the target site.

According to another example (“Example 2”), further to Example 1, the method includes deploying an embolic filter in the first branch lumen of the target site.

According to another example (“Example 3”), further to Example 2, the method includes aspirating a filter sheath of the embolic filter.

According to another example (“Example 4”), further to Example 3, the method includes removing the embolic filter after the first side branch body has been deployed.

According to another example (“Example 5”), further to any of the preceding Examples, wherein the first guide member includes a first end that is looped around a cap of the catheter.

According to another example (“Example 6”), further to any of the preceding Examples, wherein the main body further defines a second portal and a third portal operable to provide fluidic access from the main body to a second side branch and a third side branch extending from the target site when the main body is deployed at the target site, the second portal being pre-cannulated with a second guide member and the third portal being pre-cannulated with a third guide member prior to advancing the main body along the main guidewire.

According to another example (“Example 7”), further to Example 6 further includes advancing a second sheath along the second guide member through the second portal; advancing a second articulatable wire or guide catheter through the second sheath; positioning the second articulatable wire or guide catheter into a second branch lumen of the target site; advancing a third sheath along the third guide member through the third portal; advancing a third articulatable wire or guide catheter through the third sheath; and positioning the third articulatable wire or guide catheter into a third branch lumen of the target site.

According to another example (“Example 8”), further to Example 7, the method includes advancing a second side branch body along the second articulatable wire or guide catheter into the second branch lumen of the target site; deploying the second side branch body in the second branch lumen of the target site; advancing a third side branch body along the third articulatable wire or guide catheter into the third branch lumen of the target site; and deploying the third side branch body in the third branch lumen of the target site.

According to another example (“Example 9”), further to Example 8, the method further includes removing the main guidewire, the first, second, and third guide members, and the first, second, and third sheaths.

According to another example (“Example 10”), further to Example 9, wherein the catheter is removed prior to advancing the first, second, and third sheaths.

According to another example (“Example 11”), an endoprosthesis delivery system includes an elongate member having a first end and a second end; an end cap coupled to the first end of the elongate member; an endoprosthesis including a main body defining a main lumen and at least one side branch portal and at least one second body defining a secondary lumen; and at least one guide member extending through the at least one side branch portal and coupled to the end cap.

According to another example (“Example 12”), further to Example 11, the endoprosthesis delivery system further includes a constraining member constraining the main body of the endoprosthesis to the elongate member.

According to another example (“Example 13”), further to Example 12, the endoprosthesis delivery system, wherein the constraining member is operable to constrain the main body at a constrained configuration and at a partially deployed configuration, the main body having a first diameter at the constrained configuration, a second diameter at the partially deployed configuration that is greater than the first diameter, and a third diameter at a deployed configuration that is greater than the first diameter and the second diameter.

According to another example (“Example 14”), further to Example 13, the endoprosthesis delivery system, wherein the constraining member includes a first portion and a second portion, wherein the first portion and the second portion are operable to independently constrain corresponding first and second portions of the main body at the constrained configuration and the partially deployed configuration.

According to another example (“Example 15”), further to any one of Examples 11-14, the endoprosthesis delivery system further includes a sheath operable to be advanced along the at least one guide member.

According to another example (“Example 16”), further to Example 15, the endoprosthesis delivery system further includes an articulatable wire or guide catheter operable to be advanced through the sheath.

According to another example (“Example 17”), further to Example 16, the endoprosthesis delivery system further includes at least one secondary branch operable to be advanced along the articulatable wire or guide catheter and to be deployed at least partially within the at least one side branch portal.

According to another example (“Example 18”), further to Example 17, the endoprosthesis delivery system further includes a removeable filter operable to be deployed downstream from a target site of the endoprosthesis.

According to another example (“Example 19”), further to Example 18, the endoprosthesis delivery system wherein the removeable filter includes a central lumen through which the articulatable or guide catheter wire is operable to extend.

According to another example (“Example 20”), further to any one of Examples 11-19, the endoprosthesis delivery system, wherein the end cap is curved.

According to another example (“Example 21”), further to the endoprosthesis delivery system of any one of Examples 11-20, wherein the endoprosthesis delivery system is curved from the end cap through the main body.

According to another example (“Example 22”), an endoprosthesis delivery system includes an elongate member having a first end and a second end; an endoprosthesis positioned longitudinally between the first end and second end of the elongate member, the endoprosthesis including a main body defining a main lumen and a side branch portal; a guide member extending through the side branch portal; and a guide member retainer removably coupled to the elongate member at a coupling position, the guide member being coupled to the guide member retainer at a position between the side branch portal and the coupling position of the guide member retainer.

According to another example (“Example 23”), further to the endoprosthesis delivery system of Example 22, wherein the main body defines a plurality of side branch portals.

According to another example (“Example 24”), further to the endoprosthesis delivery system of either Example 22 or Example 23, further includes a plurality of guide members.

According to another example (“Example 25”), further to the endoprosthesis delivery system of any one of Examples 22-24, wherein the guide member retainer extends through loops formed at an end of each of the guide members.

According to another example (“Example 26”), further to Example the endoprosthesis delivery system of any one of Examples 22-25, wherein the guide member retainer is operable to be selectively decoupled from the first coupling position.

According to another example (“Example 27”), further to the endoprosthesis delivery system of any one of Examples 22-26, wherein the elongate member includes a lock wire retainer positioned at the first end of the elongate member.

According to another example (“Example 28”), further to the endoprosthesis delivery system of Example 27, wherein the guide member retainer is releasably coupled to the lock wire retainer.

According to another example (“Example 29”), further to the endoprosthesis delivery system of any one of Examples 22-28, further including a side branch body, wherein each guide member includes a first end, wherein each first end of the guide members is retained by the guide member retainer between the coupling position and the side branch portal when the side branch body is advanced along the guide member.

According to another example (“Example 30”), further to the endoprosthesis delivery system of any one of Examples 22-29, wherein each guide member is operable to be removed from a corresponding side branch portal when the guide member retainer is released.

According to another example (“Example 31”), further to the endoprosthesis delivery system of any one of Examples 22-30, further including a plurality of guide member retainers, wherein each guide member retainer is coupled to a corresponding guide member.

According to another example (“Example 32”), further to the endoprosthesis delivery system of Example 29, wherein each guide member retainer is operable to be individually and selectively released from engagement at the first coupling position such that each guide member is operable to be individually removed from a corresponding side branch portal.

According to another example (“Example 33”), further to the endoprosthesis delivery system of Example 22, wherein the elongate member includes a cap positioned at the first end of the elongate member, wherein the guide member retainer is coupled to the cap at the coupling position.

According to another example (“Example 34”), further to the endoprosthesis delivery system of any one of Examples 22-33, the guide member retainer is coupled to the elongate member at the first end of the elongate member.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a view of a delivery system in accordance with an embodiment;

FIG. 2 is a front view of an implantable device with a main body and side branches deployed in the aorta and adjacent side branches, in accordance with an embodiment;

FIG. 3 is a top view of a main body of an implantable device, the main body including side branch portals through which side branch bodies can be delivered and deployed, in accordance with an embodiment;

FIG. 4 is a side view of a main body of an implantable device, the main body including a portal access feature for providing clearance for side branch bodies delivered and deployed through the side branch portals in accordance with an embodiment;

FIG. 5 is an end view of a main body of an implantable device, the interior opening of the side branch portal positioned in the lumen of the main body, in accordance with one embodiment;

FIG. 6 is an end view of a main body of an implantable device, a portal access feature projecting into the lumen of the main body in accordance with one embodiment;

FIG. 7 is a perspective view of a main body including side branch portals staggered along a longitudinal length of the main body in accordance with one embodiment;

FIG. 8 is a perspective view of a main body including a side branch portal staggered relative to two side branch portals aligned along a longitudinal length of the main body in accordance with one embodiment;

FIG. 9 is a cut-away view of a patient's aorta in accordance with one embodiment;

FIG. 10 is a view of a filtration system deployed in the vasculature of a patient in accordance with one embodiment;

FIG. 11A is a view of a main body of an implantable device being delivered to a target site, the main body including side branch portals that are pre-cannulated in accordance with one embodiment;

FIG. 11B is a view of a delivery system with a guide member retainer retaining guide members via a lock wire retainer in accordance with one embodiment;

FIG. 11C is a view of a delivery system with a lock wire, the delivery system being steerable in accordance with one embodiment;

FIG. 12 is a view of a main body include a first and second region, the second region being partially deployed in accordance with one embodiment;

FIGS. 13a-13c are views of a sheath being advanced along guide members that are cannulating side branch portals of a main body in accordance with one embodiment;

FIG. 14 is a view of an articulatable guide catheter and/or wire that is advanced through a sheath and positioned in a side branch of the target lumen in accordance with one embodiment;

FIG. 15 is a view of an articulatable wire advancing through the filtration system for a through-and-through configuration between access sites in accordance with one embodiment;

FIG. 16 is a view of articulatable wires positioned in each of the side branches of the target site in accordance with one embodiment;

FIG. 17 is a view of a main body partially deployed along the full longitudinal length of the main body for accurate placement of the main body in the target lumen in accordance with one embodiment;

FIG. 18 is a view of a main body fully deployed in the target lumen in accordance with one embodiment;

FIG. 19 is view of side branch bodies being delivered to corresponding branches of a target site in accordance with one embodiment;

FIG. 20 is a view of side branch bodies being deployed at corresponding branches of a target site in accordance with one embodiment;

FIG. 21 is a view of delivery systems for side branch bodies being removed from the target site in accordance with one embodiment;

FIG. 22 is a view of a filtration system being aspirated prior to removal of delivery systems and the filtration system used for deploying an implantable device to a branched target site in accordance with one embodiment;

FIG. 23 is a view of an implantable device implanted as a branched target site prior to removal of a plurality of guidewires used to cannulate each portion of the branched target site in accordance with one embodiment;

FIG. 24 is a view of a sleeve for constraining an implantable device site in accordance with one embodiment;

FIG. 25 is a view of an implantable device with selectable fenestrations for side branches in accordance with one embodiment;

FIGS. 26A-26C are various views of a main body graft of the implantable device of FIG. 25; and

FIGS. 27A-27C are various embodiments of coupling guide members for a delivery system.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

For reference, the terms “circumference” and “diameter” are not meant to require a circular cross-section (although are inclusive of a circular cross-section), and are instead to be understood broadly to reference an outer surface or dimension and the dimension between opposing sides of the outer surface, respectively.

Although the embodiments herein may be described in connection with various principles and beliefs, the described embodiments should not be bound by theory. For example, embodiments are described herein in connection with vascular stent grafts, and more specifically branched stent grafts. However, embodiments within the scope of this disclosure can be applied toward any endoprostheses of similar structure and/or function. Furthermore, embodiments within the scope of this disclosure can be applied in non-vascular applications.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Devices, systems, and methods of endoluminally delivering a branchable expandable implant in accordance with various embodiments are disclosed herein for treating disease of human vasculature. Although the description below and figures are illustrated in the context of treating the aorta 20, including the ascending aorta 21, aortic arch 22, and descending aorta 23, and branches therefrom, including the brachiocephalic artery 24, the left common carotid artery 25, and the left subclavian artery 26, it should be appreciated that the present disclosure can be applied to treatment of other portions of the vasculature or, including, for example, any disease where a larger vessel and one or more branch vessels are to be treated.

### Branchable, Expandable Implant

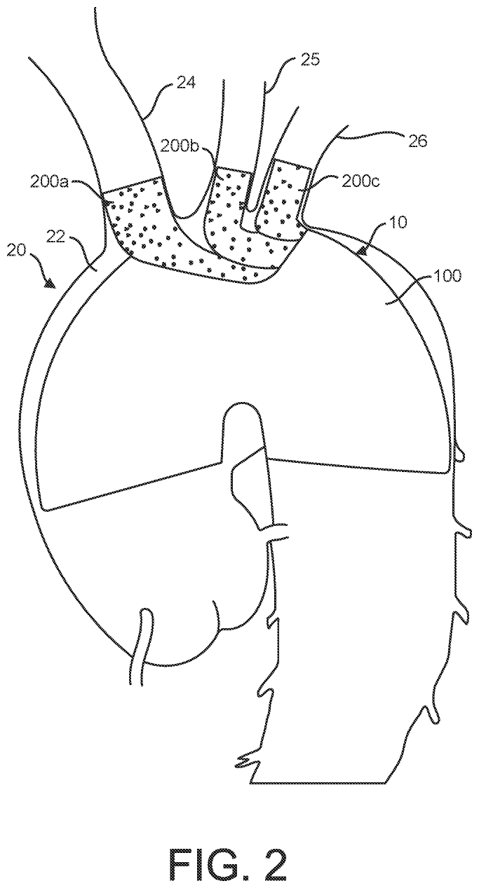

Referring to FIG. 2, an implantable device 10 can be delivered and deployed in the aorta 20, the implantable device 10 including a main body 100 and branch bodies 200. The main body 100 is deployed in the aortic arch 22 and the branch bodies 200 can be deployed in branching arteries (e.g., a first branch body 200a in the brachiocephalic artery 24, a second branch body 200b in the left common carotid artery 25, and a third branch body 200c in the left subclavian artery 26).

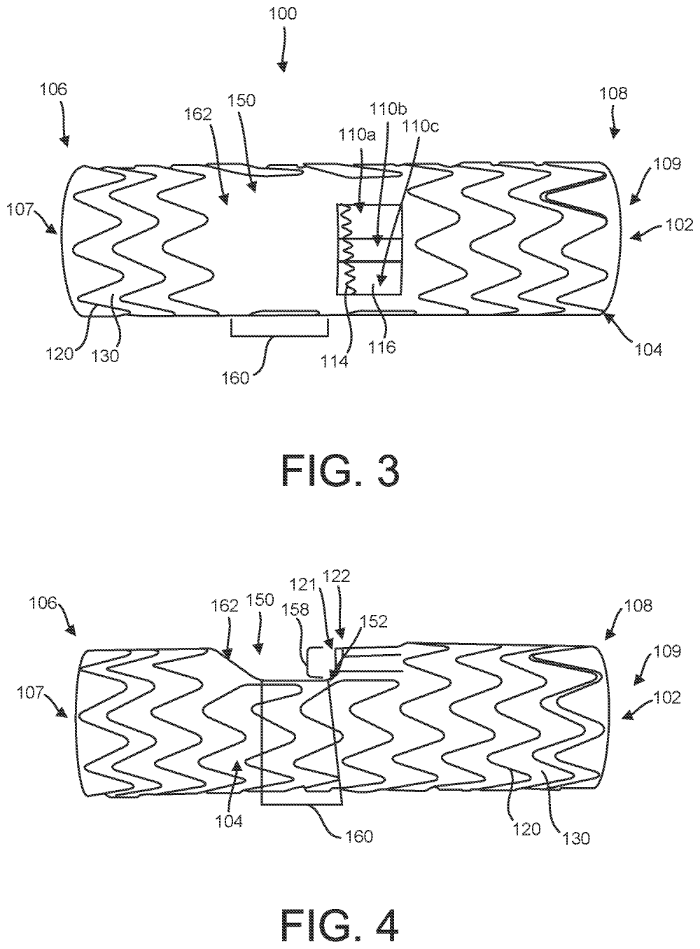

Although various configurations of the implantable device 10 are contemplated with respect to the delivery systems and methods described herein, several discrete examples of an implantable device 10 are provided in detail in order to provide reference for the various components and steps of the delivery system and method of delivery and deployment. For example, FIG. 3 is an exemplary embodiment of an implantable device 10. The main body 100 includes a wall 104 forming the main lumen 102. The main body 100 has a first end 106 and a second end 108. At the first end 106, the main body 100 includes a first opening 107 and at the second end 108 the main body 100 includes a second opening 109. Each of the openings 107, 109 provides access to the main lumen 102 at the corresponding end 106, 108. Fluids are operable to flow through the main lumen 102 by passing through the first opening 107, into the main lumen 102, and out the second opening 109, defining a main body fluid flow direction. Or, the flow may be in the opposite direction, defining the main body fluid flow direction. The outer wall 104 substantially forms or defines the outer profile of the main body 100.

In some embodiments, the main body 100 is formed of a stent structure 120 and a graft member 130. The stent structure 120 is operable to maintain patency of the main body 100 and/or the main vessel (e.g., the aorta 20) when the main body 100 is deployed. The stent structure 120 can be formed of various materials, including, but not limited to, metals, metal alloys, polymers, and any combination thereof to provide elastic or plastic properties (e.g., self-expanding or balloon-expandable stents). The graft member 130 is coupled to the stent structure 120 and forms the fluid impermeable or semi-permeable layer through which fluids may flow (e.g., blood).

The main body 100 further includes at least one side branch portal 110. The side branch portal 110 is operable to provide fluidic access between the main lumen 102 and a branch vessel. The side branch portal 110 forms or is positioned in an opening 112 through the wall 104 along the outer profile of the main body 100. In certain instances, the side branch portal 110 extends through the wall 104 of the main body 100 longitudinally between the first end 106 and the second end 108 of the main body 100. Thus, fluid may flow through the first opening 107 and through the side branch portal 110. Some embodiments include a plurality of side branch portals 110. For example, FIG. 3 illustrates a main body 100 included a first side branch portal 110a, a second side branch portal 110b, and a third side branch portal 110c. Any number of side branch portals 110 may be incorporated to accommodate the specific anatomy into which the device 10 is to be deployed.

Referring still to FIG. 3, in some embodiments, each of the side branch portals 110 includes a side branch stent structure 114 and a side branch graft member 116. In various embodiments, the side branch stent structure 114 and side branch graft member 116 can be independent from, incorporated into, or integral with the main body stent structure 120 and the main body graft member 140. For example, as illustrated in FIGS. 3-5, the side branch stent structures 114 is separate or independent from the main body stent structure 120, whereas the side branch graft member 116 is incorporated into the main body graft member 140 (e.g., sandwiched or interposed between layers of the main body graft member 140). In some embodiments, the side branch stent structure 114 extends from the main body stent structure 130 and therefore represents a portion of the main body stent structure 130 rather than an independent stent structure. In still other embodiments, the side branch stent structure 114 is coupled to the main body stent structure 130. Similarly, the side branch graft member 116 can be formed directly from the main body graft member 140 and therefore represent a portion of the main body graft member 140. In other embodiments, the side branch graft member 116 is coupled to the main body graft member 140 or, or in still other embodiment, is spaced from the main body graft member 140. It is understood that any combination of side branch stent structures 114 and side branch graft member 116 embodiments is within the scope of this disclosure.

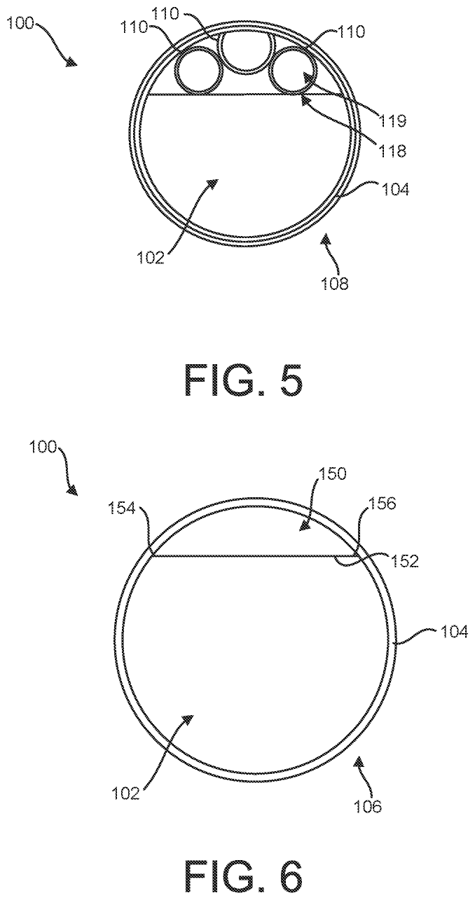

In some embodiments, the side branch portal 110 is positioned between the first end 106 and the second end 108 of the main body 100 and does not extend beyond or increase the outer profile of the main body 100 (see FIGS. 4 and 5). Stated otherwise, the portion of an outer wall of the side branch portal 110 is positioned along the wall 104 of the main body 100 within the outer profile of the main body 100 (e.g., flush with the outer profile). Thus, the side branch portal 110 may extend into the main lumen 102 of the main body 100 without substantially increasing the outer profile of the main body 100 adjacent the exit location of the side branch portal 110 from the main body 100.

Each side branch portal 110 may be include a first end 118 and a second end 122 defining a first opening 119 and a second opening 121, respectively. Fluids travel through the side branch portal from the first end 118 to the second end 122 (or vice versa) defining a side branch fluid flow direction. The side branch portal 110 is positioned such that the first opening 119 is positioned within or oriented toward the main lumen 102 of the main body 100 and the second opening 121 is positioned exterior to or oriented away from the main body 100 (e.g., the first opening 119 is the interior opening and the second opening 121 is the exterior opening of the side branch portal 110 relative to the wall 104 and main lumen 102 of the main body 100). For example, FIG. 5 illustrates those embodiments in which the first opening 119 of the side branch portal 110 is positioned within the main lumen 102. The side branch portal 110 may have various longitudinal lengths. Furthermore, when a plurality of side branch portals 110 are implemented, each side branch portal 110 may include various lengths or may be uniform in length. It is understood that in embodiments implementing a plurality of side branch portals 110, each side branch portal 110 may have an independent diameter or geometric orifice area.

In some embodiments, the side branch portal 110 is oriented such that the side branch fluid flow direction is opposite to the main body fluid flow direction (e.g., retrograde to the main body fluid flow direction). It is understood that opposite or retrograde in these embodiments is not limited to 180 degrees of difference, but generally encompasses a change in the direction of the fluid flowing that is greater than 90 degrees. It is also understood that the direction of the fluid flow is with respect to the specific location along the longitudinal length of the main body 100 as the main body may conform to a curved anatomy. For example, in embodiments where the side branch fluid flow direction is opposite or retrograde to the main body fluid flow includes those embodiments in which the side branch portal 110 second opening 121 is longitudinally closer to the main body 100 first end 106 relative to the side branch portal 110 first opening 119. By orienting the side branch portal 110 in the retrograde orientation, a surgeon may be able to perform the intervention and any subsequent interventions from a more advantageous access site (e.g., femoral access site to reduce trauma to carotid arteries, subclavian, or other arteries or decrease surgical presence in more anatomically crowded portions of a patient such as around the neck or thorax when operating in the aortic arch). This orientation may be advantageous in some presentations where access may difficult, obstructed, or dangerous from certain access sites.

In other embodiments, the side branch portal 110 is oriented such that the side branch fluid flow direction is generally oriented with the main body fluid flow direction (e.g., antegrade to the main body fluid flow direction). In embodiments where the side branch fluid flow direction is antegrade to the main body fluid flow includes those embodiments in which the side branch portal 110 first opening 119 is longitudinally closer to the main body 100 first end 106 relative to the side branch portal 110 second opening 121. Antegrade orientations may be advantageous in some embodiments to maintain more traditional fluid flow, especially in tissues or anatomies that may have unique geometries that would limit the use of a retrograde orientation. In embodiments implementing a plurality of side branch portals 110, the side branch portal may all have an antegrade orientation, may all have a retrograde orientation, or may include one or more branch portals with an antegrade orientation and one or more portals having a retrograde orientation.

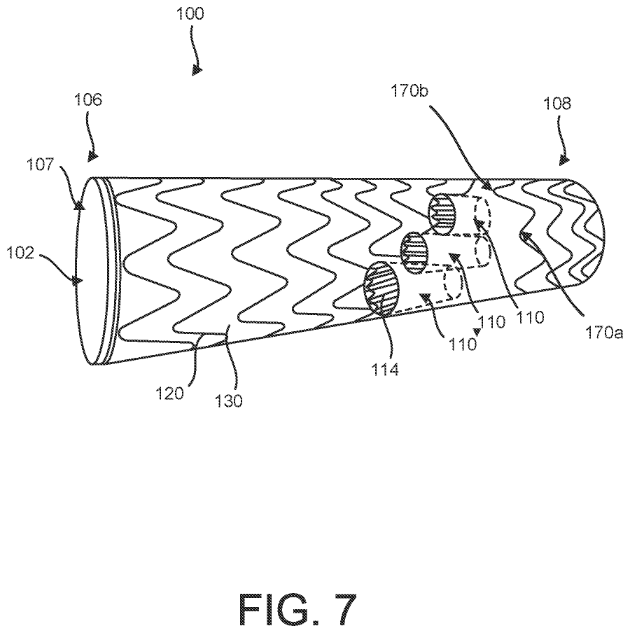

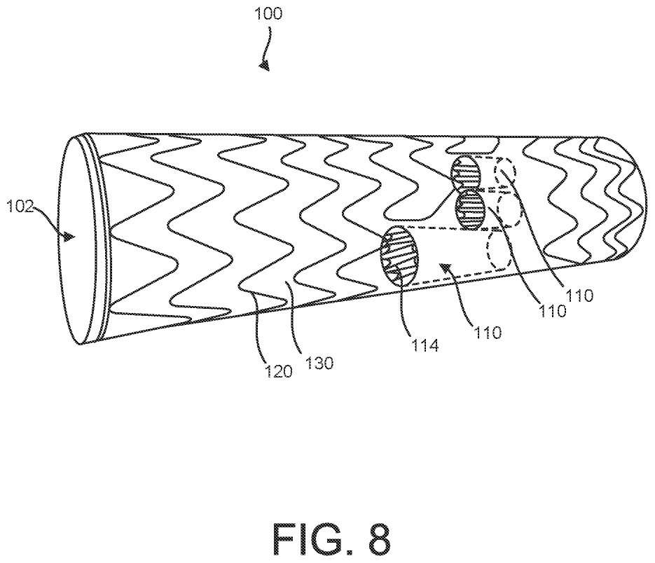

The second opening 121 of the side branch portal 110 can be positioned at various longitudinal positions between the first end 106 and the second end 108 of the main body 100. For example, the second opening 121 of the side branch portal 110 may be positioned generally at the midpoint between the first and second ends 106, 108 of the main body 100. In other embodiments, the second opening 121 of the side branch portal 110 may be positioned closer to the first end 106 relative the second end 108 or, alternatively, closer to the second end 108 relative to the first end 106 of the main body 100. In those embodiments including a plurality of side branch portals 110, each second opening 121 may be aligned longitudinally along the length of the main body 100 (see FIG. 3), staggered along the length of the main body 100 (see FIG. 7), or a combination thereof (see FIG. 8).

The side branch portals 110 may be incorporated into the main body 100 in variety of ways. For example, the side branch portals 110 may be wrapped between layers of film in the graft member 130. It is noted that in those embodiments in which a plurality of side branch portals 110 are implements, a plug (not shown) may be inserted into any one or multiple side branch portals 110 if one or more of the side branch portals are not needed in a particular application. For example, a device 10 may include three side branch portals 110, but only two are needed fora patient (e.g., in the aortic arch with a bypass), one of the side branch portals 110 may be closed (e.g., via a plug).

In some embodiments, the stent structure 120 extends around an outer periphery of the side branch portals 110. In embodiments implementing a side branch stent structure 114 that may implement materials that are more discreet or provide less holding or expansion force than the main body stent structure 120, the stent structure 120 may extend around the side branch portals 110 to limit collapsing of the side branch portals 110 (and side branch stent structures 114 when included) during delivery, deployment, and used of the device 10. However, in some embodiments, the stent structure 120 does not extend around the side branch portals 110.

Referring now to FIG. 4, the main body 100 includes a portal access feature 150. The portal access feature 150 is operable to provide clearance for branch bodies 200 that are at least partially positioned and deployed within the side branch portal 110. For example, the portal access feature 150 may be a portion of the wall 104 of the main body 100 that has a recessed outer profile. For example, in FIG. 4, the main body 100 as illustrated includes a substantially circular cross-section along the longitudinal length of the main body 100 except at the longitudinal lengths of the main body 100 defining the portal access feature 150. FIG. 5 illustrates the main body 100 from a side view looking through the main lumen 102. In this view, the substantially circular outer profile is illustrated. This view also depicts the profile of the main body at the portal access feature 150. The main body 100 at the portal access feature 150 includes a cross-section that is substantially circular with a truncated or chord portion 152 of the wall 104 extending from a first position 154 of the wall 104 across to a second position 156 of the wall 104. As illustrated, the portal access feature 150 deviates from the typical outer profile of the remainder of the main body 100 such that the portal access feature 150 appears to be radially inward from the remainder of the main body 100.

Referring again to FIG. 4, the portal access feature 150 is defined in the wall 104 of the main body 100 from at least the second opening 121 of the side branch portal 110 toward the first end 106 of the main body. The depth 158 of the portal access feature 150 is substantially equal to diameter of the side branch portal 110. The portal access feature 150 may extend from the second opening 121 of the side branch portal 110 at the depth 158 for a predetermined length to define the entry portion 160. The predetermined length of the entry portion 160 can provide sufficient space for the branch body 200 to exit the side branch portal 110 and turn or bend toward the branch vessel and defines an entry portion 160 of the portal access feature 150. The entry portion 160 in some embodiments is substantially flat, as is illustrated in FIG. 4. However, the entry portion 160, in some embodiments, can incorporate a curvature. For example, in some embodiments, the entry portion 160 includes an arcuate profile. The arcuate profile may allow a plurality of side branch portals 110 to be implemented (e.g., each side branch portal 110 having the same diameter), where a bottom edge of each side branch portal 110 aligns with the entry portion 160 of the portal access feature 150 and the top edge aligns with the outer profile of the main body 100 (not shown). The portal access feature 150 may also include a transition portion 162. The transition portion 162 includes the portion of the wall 104 that transitions into the entry portion 160. The transition portion 162 may also be operable to accommodate the branch body 200 as it exits the side branch portal 110. In some embodiments, the transition portion 162 extends directly from the second opening 121 of the side branch portal 110 (not shown). In still further embodiments, the portal access feature 150 is a narrowing (not shown) of the main body 100 proximate the second opening 121 of the side branch portal 110.

It is understood that the portal access feature 150 does not have to begin at the second opening 121 of the side branch portal 110. For example, in some embodiments, the portal access feature 150 extends beneath the side branch portals 110. The side branch portals may be positioned between the portal access feature 150 and an outer layer of the graft member 130. In these embodiments, the portal access feature 150 extends from the side branch portal 110 toward the first end 106 of the main body 100.

With further reference to FIG. 4, the portal access feature 150, in some embodiments, the portal access feature is free of any stent. In some embodiments, the stent structure 120 used to support the graft member 130 does not extend onto the portal access feature 150. For example, in those embodiments in which the stent structure 120 is helically wound, the stent structure 120 does not extend across the portal access feature 150, but instead has a longitudinal portion that extends along the length of the main body 100 proximate the portal access feature 150 and extends away from the portal access feature 150 at each end of the longitudinal portion. It is understood that the stent structure 120 can include various features such as apices 170, sinusoidal shapes and so forth while generally still being helically wound. In other embodiments, the stent structure 120 may include a plurality of independent rings that are longitudinally spaced along the length of the main body 100. The rings of the stent structure 120 that are positioned at a shared longitudinal length of the main body 100 with the portal access feature 150 may terminate proximate the portal access feature 150 instead of extending fully around the main body 100, or may include a longitudinal portion that connects rings as discussed with respect to helical winding.

In other embodiments, the stent structure 120 can extend across the portal access feature 150. For example, in an embodiment in which the stent structure 120 extends across the portal access feature 150, the stent structure can be formed and/or shape set to accommodate and/or form the profile of the portal access feature 150. The portion of the stent structure 120 defined over the portal access feature 150 may be continuous with the remainder of the stent structure 120. For example, in main bodies 100 implementing a stent structure 120 that is helically disposed or wrapped about the main body 100, the stent structure 120 may substantially continue the helical path at the portal access feature 150. In some embodiments, the apices 170a of the stent structure 120 at the portal access feature 150 may be shorter than the apices 170b around the remainder of the main body 100 (see FIG. 7). Furthermore, the frequency may be decreased such that more apices are incorporated into a circumferential length of the main body 100 at the portal access feature 150. In other embodiments, the stent structure 120 positioned at the portal access feature 150 is shaped to outline or otherwise conform to the peripheral profile of the portal access feature 150. In these embodiments, the stent structure 120 of the portal access feature 150 extends from or is coupled to the stent structure 120 of the remainder of the main body 100, but has a shape independent from or not conforming to the pattern of the stent structure 120 of the remainder of the main body 100.

In some embodiments, the portal access feature 150 may include a portal access stent (not shown) that is independent from the stent structure 120 as previously discussed. The independent stent member can is coupled to the graft member 130 at the portal access feature 150. The independent stent member can incorporate any number of configurations, including patterns operable to conform to the peripheral profile of the portal access feature 150.

The portal access feature 150 may further include a reinforcing material. The reinforcing material is operable to provide increased strength to the portal access feature 150. The reinforcing material can resist tear, puncture, and other damage that can be incurred by the portal access feature 150 as the device 10 is being deployed. For example, cannulation and/or delivery and deployment of the branch body 200 may result in contacting the portal access feature, the reinforcing material being sufficiently sturdy to withstand tears or wear that could result in damage to the device 10. In some embodiments, the reinforcing material is applied to the portal access feature, is incorporated into the graft member 130 at the portal access feature, or a combination thereof. Various materials may be implemented for the reinforcement material, including but not limited to dense ePTFE layers or multilayers.

### Delivery System and Methods of Delivery and Deployment

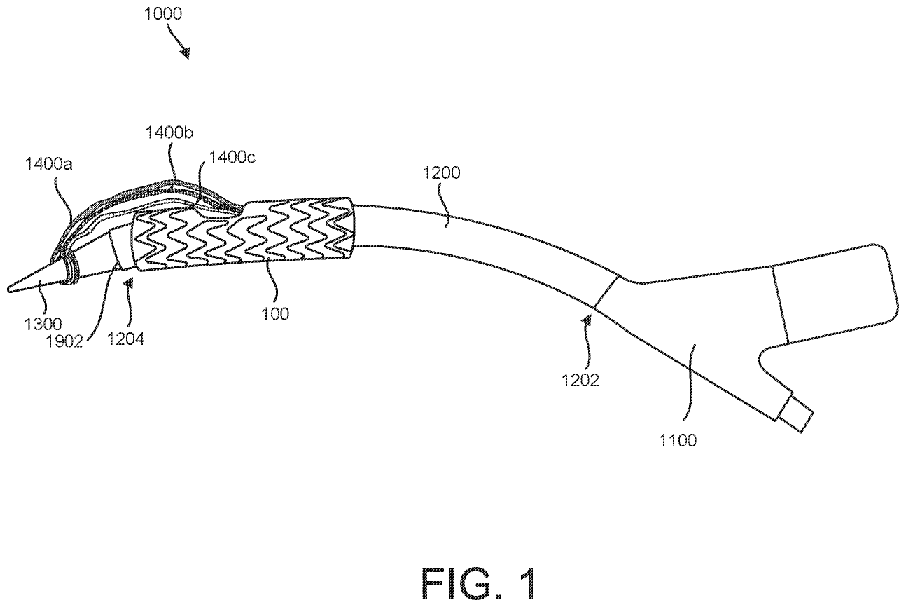

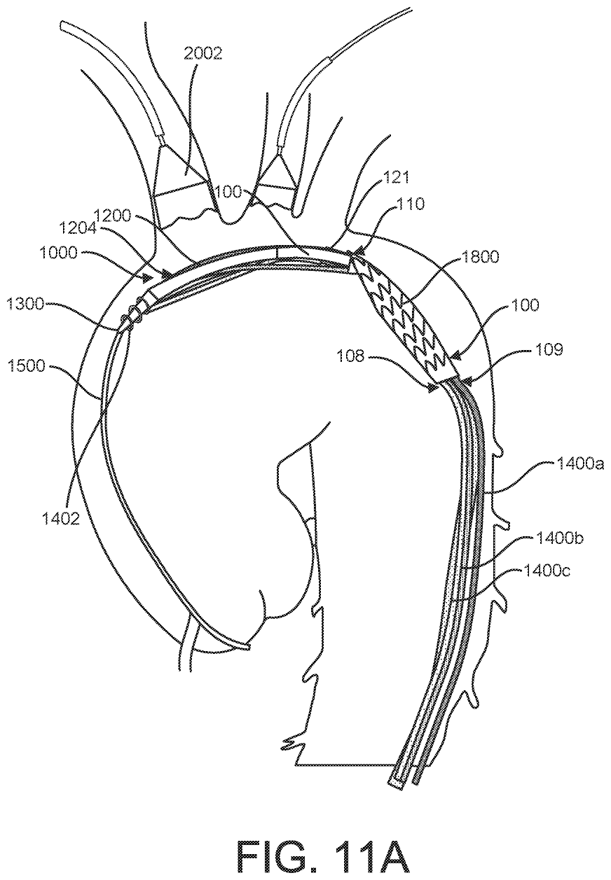

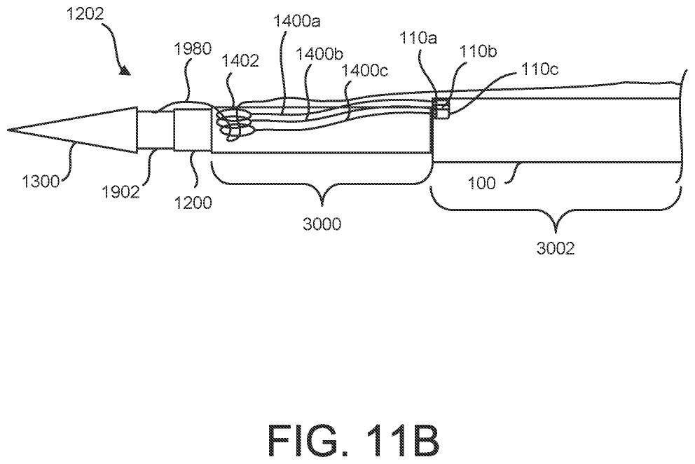

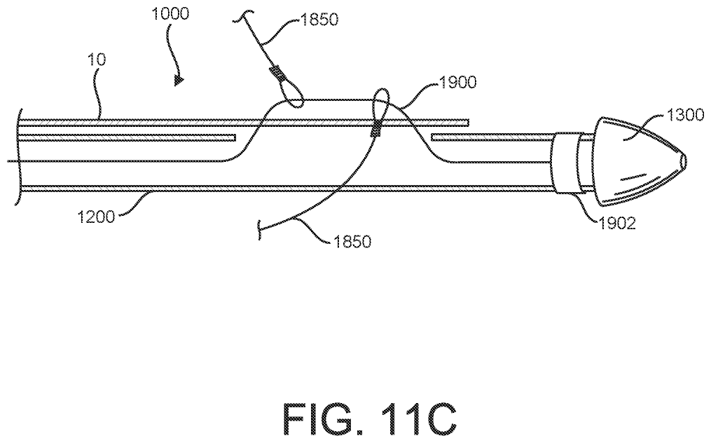

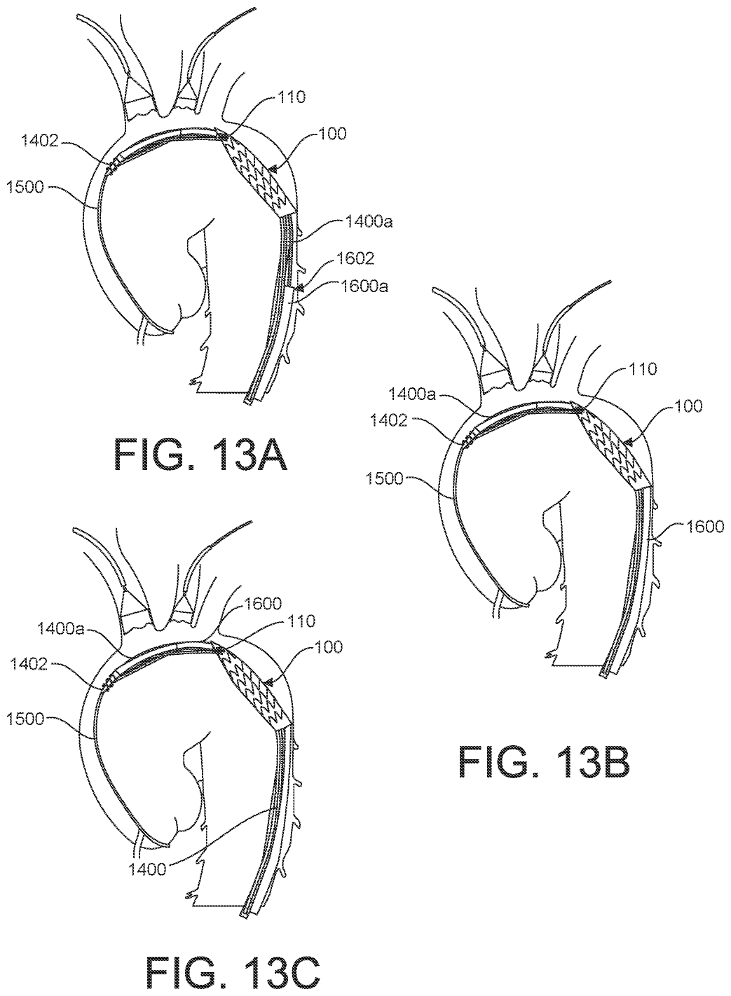

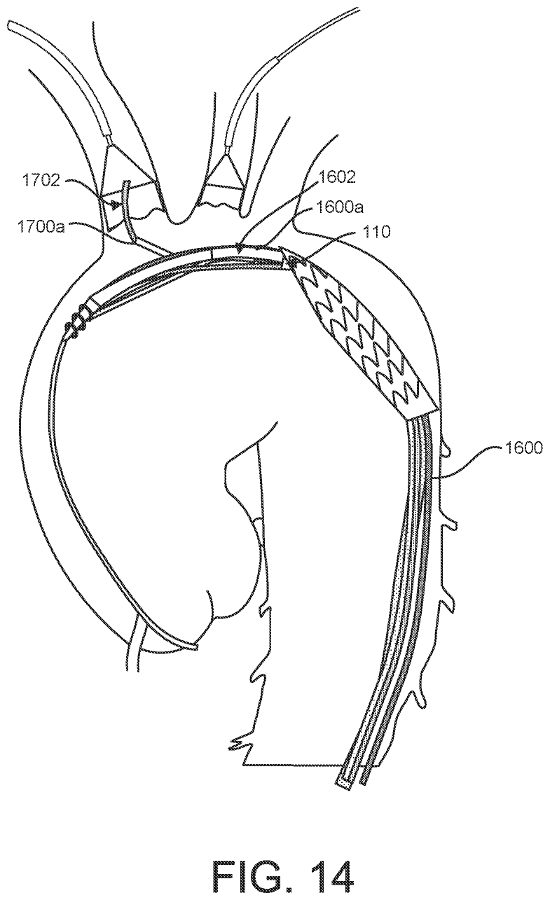

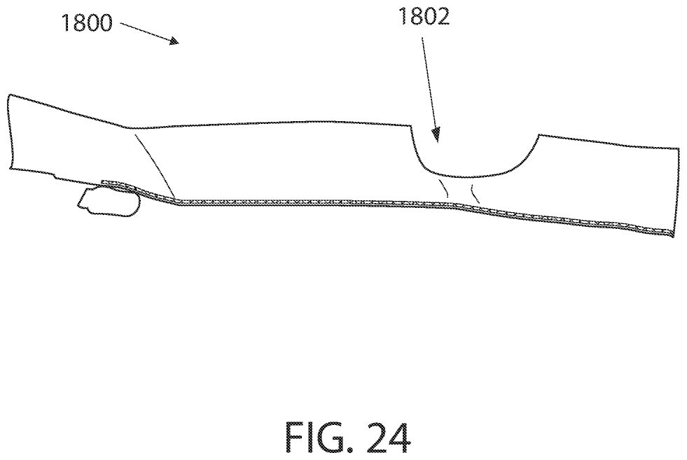

Referring to FIG. 1, a delivery system 1000 is illustrated (not necessarily to scale). The delivery system 1000 is operable to deliver a multi-component implantable device (e.g., implantable device 10) to a target site. The delivery system includes a handle 1100, an elongate member 1200 having a first end 1202 coupled to and/or extending from the handle 1100 and a second end 1204, a cap 1300 positioned proximate the second end 1204 of the elongate member 1200, and at least one guide member 1400 extending at least partially along the elongate member 1200 toward the cap 1300. The elongate member 1200 and cap 1300 are operable to translate along a main guidewire 1500 (see FIG. 10). The delivery system 1000 can further include at least one sheath 1600 (see FIGS. 13a-13c), the sheath 1600 operable for use with the guide member 1400 (when there are a plurality of guide members 1400a, 1400b, 1400c, each guide member 1400 has a corresponding sheath 1600). An articulatable secondary guidewire 1700 (see FIG. 14) can be included for each sheath 1600. The delivery system 1000 may also include a constraining member 1800 (see FIG. 11) that is operable to constrain at least a portion of a multi-component implantable device. It is understood that an individual constraining member 1800 may be implemented for each discrete component of the multi-component implantable device. The delivery system 1000 can be used in conjunction with filtration systems 2000 (e.g., to reduce risk of embolism, see FIG. 10). In some embodiments, the constraining member 1800 may include a window 1802 for the side branch portals 110, the window 1802 of the constraining member 1800 positioned overlaying the side branch portals such that the side branch portals 110 are accessible when the constraining member 1800 is constraining the device 10 (see FIG. 24).

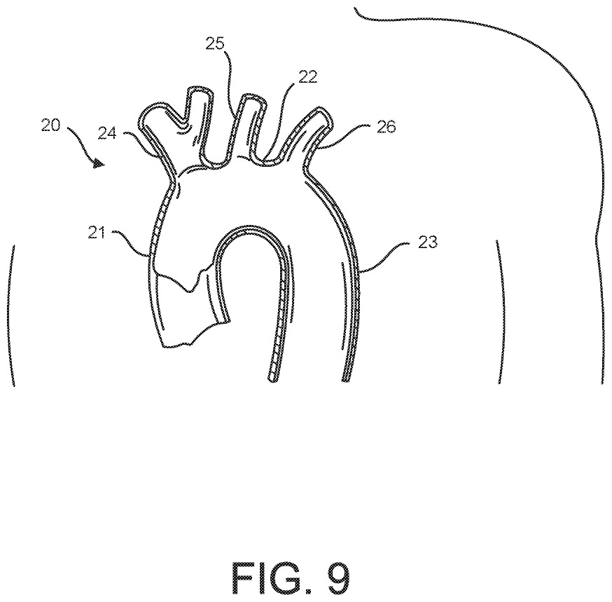

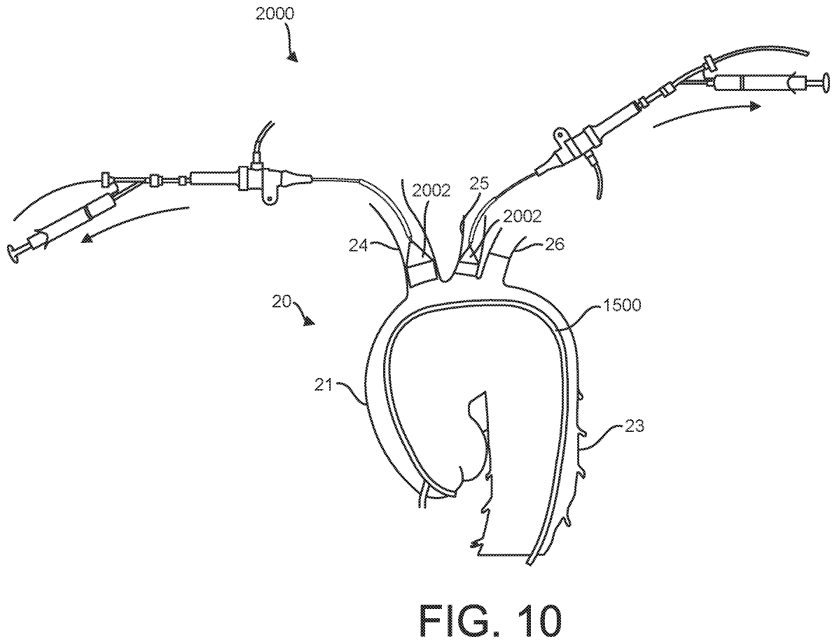

Referring to FIG. 9, an exemplary target site for delivery and deployment of a multi-component implantable device is illustrated. In this example, the aorta 20 is illustrated. However, it is understood that the delivery system 1000 may be implemented in any part of the vasculature that includes branched lumens as appropriate. In this example, the aorta 20 is illustrated, including the ascending aorta 21, aortic arch 22, and descending aorta 23, and branches therefrom, including the brachiocephalic artery 24, the left common carotid artery 25, and the left subclavian artery 26.

FIG. 10 illustrates implementation of the filtration system 2000 in connection with the delivery system 1000. The filtration system 2000 can include a plurality of deployable filters 2002 that can be deployed in discreet lumens, including side branch lumens, that are fluidically downstream from the target site at which the multi-component implantable device is to be implanted. The filtration system 2000 can be intermittently flushed throughout the procedure. A main guidewire 1500 is advanced to the target site (e.g., the aortic arch). Although the main guidewire 1500 is illustrated as coming from the descending aorta 23 (e.g., from a femoral access site), the main guidewire 1500 can be inserted from any appropriate access site.

Referring to FIG. 11, a multi-component implantable device is advanced to the target site via the guidewire 1500. For the purposes of the example provided herein, the multi-component implantable device will include the embodiment disclosed with respect to FIGS. 3-4. However, it is understood that the methods and the delivery system 1000 are not limited to delivering only the implantable device 10 as described with reference to FIGS. 3 and 4. The implantable device (e.g., the main body 100) is positioned on the elongate member 1200. For example, the implantable device 10 may be constrained in a compressed configuration about the elongate member 1200. For example, the implantable device 10 can be constrained by a constraining member 1800. The implantable device 10 may be positioned proximate the cap 1300 and the second end 1204 of the elongate member 1200.

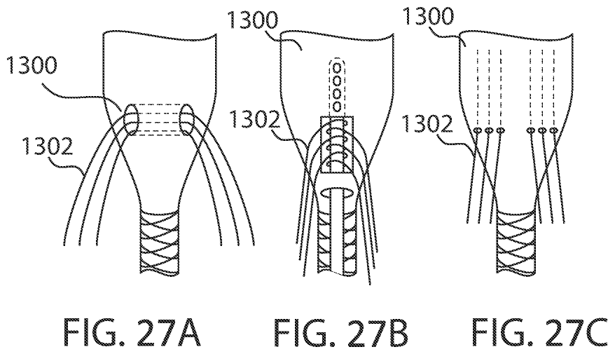

As is illustrated in FIG. 11A, the delivery system 1000 may include a plurality of guide members 1400a, 1400b, 1400c. The guide members 1400a, 1400b, 1400c are coupled (e.g., releasably coupled) to the delivery system 1000 proximate the second end 1204 of the elongate member 1200. For example, in some embodiments, the guide members 1400a, 1400b, 1400c are coupled to the cap 1300. The guide members 1400a, 1400b, 1400c may each form a loop 1402 (one of which is referenced in FIG. 11A for ease of illustration) which can be fastened to or disposed about at least a portion of the cap 1300. In other embodiments, the guide members 1400a, 1400b, 1400c may implement a coupling system (not shown) for coupling the guide members 1400 to the to the delivery system 1000 proximate the second end 1204 of the elongate member 1200, the coupling system including a feature, for example a ball tip, that is received by a corresponding member proximate the second end 1204 of the elongate member 1200 (e.g., the cap 1300 positioned proximate the second end 1204 of the elongate member 1200 may include a corresponding member). Other examples of embodiments for matingly engaging or coupling the guide members 1400a, 1400b, 1400c at an end of the delivery system 1000 proximate the second end 1204 of the elongate member 1200 can be achieved by a variety of coupling arrangements, including press fitting, threads, ball and detent, articulating clips or jaws, hook and loop, and magnetic. Any number of methods and structures may be implemented for fastening the guide members 1400a, 1400b, 1400c proximate the second end 1204 of the elongate member 1200, and the disclosed embodiments are not to be limiting to the scope of the disclosure. It is also understood that the guide members 1400 can be fastened at various other positions on the delivery system 1000. For example, in some embodiments, the guide member 1400 may be fixed to the elongate member 1200 or other portions of the delivery system 1000. In some embodiments, the guide members 1400 are fastened to an internal wall of the main body 100 (e.g., via a releasable suture). In some embodiments, the guide members 1400 can be retained at a position via a lock wire retainer 1902, which is described in further detail hereafter. The lock wire retainer 1902 may be implemented solely for capturing the guide members 1400 or may be used in connection with other members for various other purposes, including but not limited to steering and positioning the main body 100 at the target site, which will be described hereafter. Further examples for coupling the guide members 1400 with the delivery system 1000 are provided hereafter and are discussed with regard to FIGS. 27A-27C.

Referring to FIG. 11B, in some embodiments, a guide member retainer 1980 can be implemented with respect to the guide members 1400 in order to retain the guide members 1400 during delivery of the implantable device 10 and advancement of the sheaths 1600 along the guide members 1400. For example, as illustrated in FIG. 11B, the delivery system 1000 includes an elongate member 1200 having a first end 1202. At the first end 1202, the delivery system includes a lock wire retainer 1902 and an end cap 1300 (e.g., the lock wire retainer 1902 is positioned between the end cap 1300 and the first end 1202 of the elongate member 1200). The main body 100 is positioned about the elongate member 1200 where the second region 3002 of the main body 100 is partially deployed and the first region 3000 is constrained. Guide members 1400 are extending through the side branch portals 110 toward the first end 1202 of the elongate member 1200. The guide members 1400 include a retaining member at the end of each guide member 1400 (e.g., loops 1402). The guide member retainer 1980 is releasably coupled to the lock wire retainer 1902 and extends along the elongate member 1200. The guide member retainer 1980 is operable to retain the guide members 1400 at or proximate the first end 1202 of the elongate member 1200 (e.g., at the lock wire retainer 1902, proximate the cap 1300, etc.). The guide member retainer 1980 may be releasably coupled to the delivery system 1000 at a coupling position, for example, releasably and selectively coupled to the lock wire retainer 1902. The guide member retainer 1980 may capture, lasso, or otherwise retain the guide members 1400 at a longitudinal position relative to the elongate member 1200 such that the guide members 1400 are restricted from being retracted along the longitudinal length of the elongate member 1200. For example, the guide member retainer 1980 is fixedly coupled to an end of each of the guide members 1400 (e.g., the loop 1402) such that the guide members 1400 are restricted from retracting when the guide member retainer 1980 is engaged with the lock wire retainer 1902. The position where the guide member retainer 1980 is engaged with the guide members 1400 is generally at a position between the lock wire retainer 1902 and the side branch portals 110.

In some embodiments, the guide members 1400a, 1400b, 1400c may implement a coupling system for coupling the guide members 1400 to the to the guide member retainer 1980 proximate the second end 1204 of the elongate member 1200, the coupling system including a feature, for example a spherical tip, that is received by a corresponding member of the guide member retainer 1980. For example, the guide member retainer 1980 may receive the spherical tip of the guide members 1400 through an aperture or through a loop, where the diameter of the spherical tip of the guide members 1400 is greater than the diameter of the aperture or loop of the guide member retainer 1980. Other examples of embodiments for matingly engaging or coupling the guide members 1400a, 1400b, 1400c at an end of the delivery system 1000 proximate the second end 1204 of the elongate member 1200 can be achieved by a variety of coupling arrangements, including press fitting, threads, ball and detent, articulating clips or jaws, hook and loop, and magnetic arrangements. Any number of methods and structures may be implemented for fastening the guide members 1400a, 1400b, 1400c proximate the second end 1204 of the elongate member 1200, and the disclosed embodiments are not to be limiting to the scope of the disclosure. In some embodiments, a plurality of guide member retainers 1980 may be implemented, each guide member retainer 1980 being operable to retain a corresponding guide member 1400. Thus, each guide member 1400 may be independently retained and released from engagement proximate the first end 1202 of the elongate member 1200. When the guide members 1400 are released, the guide members can be removed from the corresponding side branch portal 110.

In some embodiments, the guide members 1400 may be coupled directly to the lock wire retainer 1902. The guide member retainer 1980 and the guide members 1400 (either directly or indirectly from the lock wire retainer 1902) may be selectively released from the lock wire retainer 1902. Each of the guide members 1400 may be selectively retained either collectively or individually.

Now referring to FIG. 11C, in various embodiments, the delivery system 1000 may include a lock wire 1900. In such embodiments, the lock wire 1900 may secure a steering line or lines 1850 to the catheter assembly. For example, with reference to FIG. 11C, delivery system 1000 comprises an elongate member 1200, an implantable device 10, at least one steering line 1850, and a lock wire 1900. The lock wire 1900 passes from outside of the body of the patient, through the elongate member 1200, and exits at a point near a cap 1300. In some embodiments, at this point the lock wire 1900 interacts with the steering lines 1850, then reenters the elongate member 1200 and continues to the cap 1300. In some embodiments, the lock wire 1900 is coupled to a lock wire retainer 1902 (see also FIG. 1) that is positioned at the second end 1204 of the elongate members 1200, for example, between the cap 1300 and the implantable device 10. In such a configuration, the lock wire 1900 releasably couples the steering lines 1850 to delivery system 1000. Any manner in which the lock wire 1900 may interact with the steering line or lines 1850 to maintain a releasable coupling between the steering line or lines 1850 and delivery system 1000 is within the scope of the present disclosure.

In various embodiments, each steering line may further include an end loop. For example, each steering line 1850 comprises an end loop. The lock wire 1900 may pass through each end loop, securing each steering line 1850 to delivery system 1000. Any method of securing the steering line or lines 1850 to delivery system 1000 is within the scope of the invention.

In various embodiments, lock wires can be formed from metallic, polymeric or materials and can include conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol. Elongated members or lock wires can also be formed from high strength polymer fibers such as ultra high molecular weight polyethylene fibers (e.g., Spectra®, Dyneema Purity®, etc.) or aramid fibers (e.g., Technora®, etc.).

In various embodiments, a catheter assembly used to deliver an expandable implant comprises a catheter shaft, an expandable implant, one or more sleeves, one or more steering lines, and a lock wire. In such configurations, the expandable implant is capable of bending, through tension applied to the one or more steering lines and corresponding displacement, to conform to curvature in the vasculature of a patient. Tension can be applied to the steering lines 1850, causing expandable implant implantable device 10 to bend in a desired manner. For example, implantable device 10 can bend in a direction aligned with the location of the steering lines 1850. Once the implantable device 10 has been sufficiently bent, consistent tension is applied to steering lines 1850 to maintain the degree of bending. In other examples, the device 10 is configured to remain curved following tensioning of the steering lines 1850 absent a straightening force.

In various embodiments, tension can be applied to the steering lines 1850 by pulling the lines from the outside of the body of the patient. In other embodiments, the steering lines 1850 can be connected to one or more dials or other mechanisms for applying the tension at the trailing end of the elongate member 1200. In this configuration, the dial can be used to apply a desired tension, as well as maintain the correct amount of tension once a desired angle of bending of implantable device 10 has been achieved. Various embodiments may also comprise an indicator, scale, gradient, or the like which demonstrates the amount of tension or displacement of the steering line, and/or the amount of bending in implantable device. In various embodiments, the catheter assembly can comprise one more additional markings (e.g., on a handle) that allow a user to determine the orientation of the steering line with respect to the vasculature.

After a sufficient degree of bending has been achieved in the implantable device 10, the implant can be rotated for final positioning in the treatment area of the vasculature. In various exemplary embodiments, the lock wire 1900 is engaged with the steering lines 1850 such that torsional rotation of the catheter shaft causes the implantable device 10 to rotate within the vasculature. However, any configuration of the delivery system 1000 which allows for rotation of implantable device 10 is within the scope of the present disclosure.

After the implantable device 10 is in position and expanded within the vasculature, the lock wire 1900 can be disengaged from delivery system 1000. In various embodiments, the lock wire 1900 is disengaged by applying sufficient tension to the lock wire 1900 from outside of the body of the patient. After the lock wire 1900 is disengaged, the steering lines 1850 can be released from coupling with the elongate member 1200 and can be removed from implantable device 10 and delivery system 1000.

With further reference to FIG. 11A, the guide members 1400a, 1400b, 1400c are each extending through a respective side branch portal 110 of the main body 100 of the implantable device 10. Cannulation of the side branch portals 110 occurs prior to insertion of the implantable device 10 into the patient via the access site. Pre-cannulation can shorten the procedure time and simplify the steps performed during the operation, which can reduce trauma to the patient's tissue and damage to the implantable device 10. The guide members 1400a, 1400b, 1400c extend through the side branch portals 110 and through the second opening 109 of the main body 100 of the implantable device 10. Thus, the guide members 1400a, 1400b, 1400c may be positioned inside the main lumen 102 of the implantable device 10 from the side branch portals 110 to the second end 108 of the device. The guide members 1400a, 1400b, 1400c extend from the second opening 109 and toward the second end 1204 of the elongate member 1200. In some embodiments, the guide members 1400a, 1400b, 1400c are routed through the handle 1100 (see FIG. 1) and in other embodiments, the guide members 1400a, 1400b, 1400c are routed through other ports (not shown). The guide members 1400a, 1400b, 1400c may extend along the outside of the elongate member 1200, or the guide members 1400a, 1400b, 1400c may extend through the elongate member (not shown). In order to reduce tangling or crossing of the guide members 1400a, 1400b, 1400c, wire management devices (not shown) may be implemented. For example, a wire management device may be provided to minimize interaction of each of the plurality of guidewires and/or guide members with each other and other components of the delivery system 1000 in order to limit or prevent tangling, tying, or interference of the guidewires and/or guide members one with another and other components of the delivery system 1000, which obstructs advancement of devices along the guidewires and/or guide members. The wire management device maintains each of the guidewires and/or guide members in predetermined positions. The wire management device is operable to release portions of the guidewires and/or guide members when a device is advanced along the longitudinal length of the wire management device, allowing the device and its branches to be advanced through the lumen of the patient. For example, the delivery system 1000 may include a wire management device that releasably contains a plurality of guidewires and/or guide members. The wire management device may be configured to release a first portion of the at least one of the guidewires and/or guide members when a device is advanced along the main guidewire 1500 and configured to release a second portion one of the guidewires and/or guide members when the device is advanced along the main guidewire 1500 to a second longitudinal position. Thus, the wire management device progressively (described also as step-wise, inch-by-inch, or sequentially) releases the guidewires as a device is advanced with respect to the delivery system 1000. This allows the guidewires to be appropriately positioned and to interact with the device (e.g., pass into an internal lumen of the device) in accordance with delivery of the device.

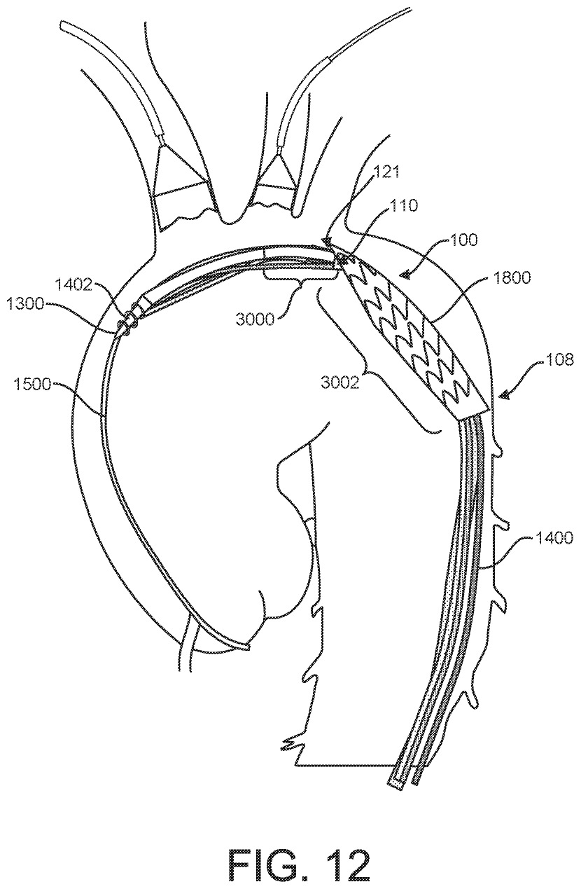

Referring now to FIG. 12, the implantable device 10 can be at least partially deployed. For example, the main body 100 can be partially deployed from a first, constrained diameter to a second, partially constrained diameter that is larger than the first diameter. As is illustrated in FIG. 12, the main body 100 can also include a first region 3000 and a second region 3002. The first region 3000 extends from the first end 106 to the second opening 121 of the side branch portal 110 and the second region 3002 extends from the second opening 121 of the side branch portal 110 to the second end 108 of the main body 100. The dividing point between the first and second regions 3000, 3002 may be defined at slightly different positions (e.g., generally within about 3 cm of the side branch portals 110). In some embodiments, the first and second regions 3000, 3002 can be independently constrained and/or deployed. For example, as illustrated in FIG. 12, the second region 3002 is partially deployed to the second, partially constrained diameter whereas the first region 3000 is maintained at the first, constrained diameter. By partially deploying the second region 3002, the side branch portals 110 are operable to at least partially expand. Such constraining members and staged deployment may include, but are not necessarily limited to primary and secondary sleeves of the constraining member 1800. The primary and secondary sleeves may be used in series which allows for expansion or partial expansion of a portion of the main body 100 by releasing one of the primary or secondary sleeves. This permits access through the side branch portals, while still allowing the main body 100 to be manipulated relative to the target site. Furthermore, by maintaining the first region 3000 in the first, constrained configuration, access through the second opening 121 of the side branch portal 110 is unrestructured or unblocked by the first region 3000 of the main body 100. This also facilitates access to the branched lumens (e.g., brachiocephalic artery 24).

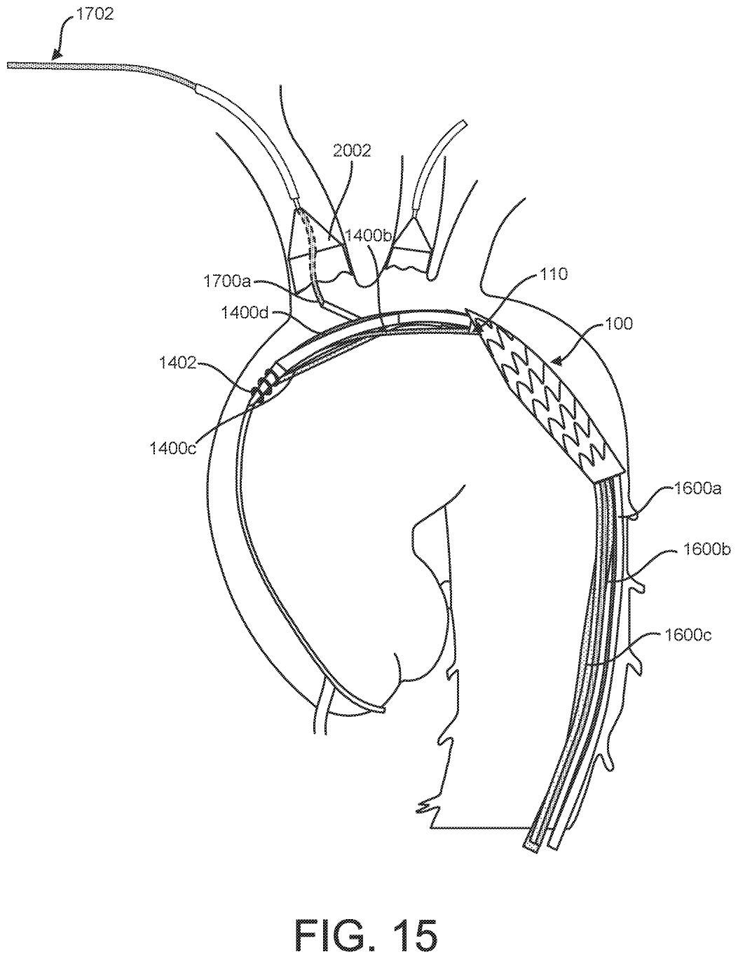

Referring now to FIGS. 13a-13c, a sheath 1600 is provided for each guide member 1400. For example, when there is a first, second, and third guide member 1400a, 1400b, 1400c for the first, second, and third side branch portals 110a, 110b, 110c, a first, second, and third sheath 1600a, 1600b, 1600c (see FIG. 15) is provided for each corresponding side branch body 200 and guide member 1400. Each sheath 1600 is operable to advance along each corresponding guide member 1400. The sheath 1600 can be formed to move along the guide member 1400 by surrounding the guide member 1400, by using the guide member as a rail in a side-by-side orientation, or as would otherwise permit the sheath 1600 the move substantially along the path of the guide member 1400. Because the side branch portals 110 are already pre-cannulated with the guide members 1400a, 1400b, 1400c, the sheath 1600 can be advanced through the second opening 109 of the elongate member and out the second opening 121 of the side branch portal 110. For example, a first end 1602 of the sheath 1600 can be advanced through the vasculature of a patient and out the second opening 121 of the side branch portal 110. The first end 1602 may be positioned proximate the corresponding branch of the vasculature (e.g., the brachiocephalic artery).

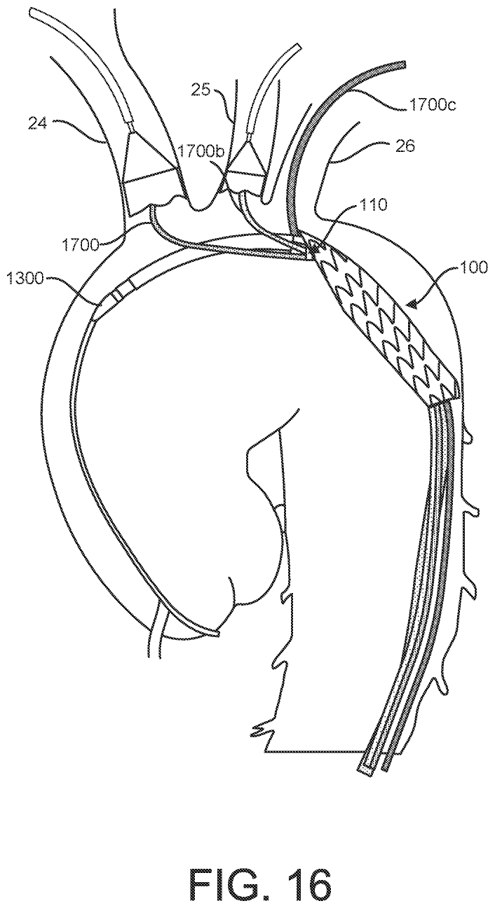

In some embodiments, the sheath 1600 includes a lumen through which an articulatable secondary guide member or catheter 1700 can be inserted (e.g., the same lumen through which the guide members 1400 are passed). Various secondary articulatable member or catheter 1700 may be implemented, including, but not limited to, steerable catheters and guidewires. For example, articulatable secondary member or catheter 1700 can be steered using at least one tether or tension member (not shown) coupled to a distal end of the articulatable guidewire 1700 (the articulatable guide member or catheter 1700 may be an integral unit, or may be a composite of various components for providing the articulating function, e.g., a guide catheter and a guidewire). The articulatable guide member or catheter 1700 may be steered by applying tension to the tether or tension member. Various degrees of motion can be achieved using multiple tethers and/or tension members. Other embodiments may include robotic or motor-driven guidewires. Various embodiments of an articulatable guidewire may be implemented in the delivery system 1000 and method. The articulatable secondary guide member or catheter 1700 is advanced to the treatment site via the sheath 1600. For example, as illustrated in FIG. 14, a first articulatable secondary guide member or catheter 1700a is advanced to the target site via the first sheath 1600a through first side branch portal 110a. The articulatable secondary guidewire 1700 includes a leading end that can be articulated by the user at a trailing end (not shown). The leading end can bend or articulate to various configurations and positions. Once the leading end of the first articulatable secondary guide member or catheter 1700a is free from the first sheath 1600a, the user is able to articulate the leading end of the first articulatable secondary guide member or catheter 1700a into position in the target branch corresponding to the first side branch portal 110a (e.g., the brachiocephalic artery). Once the secondary guide member or catheter 1700a is in place, a guidewire 1702 may be advanced into position (e.g., through the secondary guide member or catheter 1700a). This step is repeated for each branch and corresponding side branch portal 110 with a subsequent articulatable secondary guide member or catheter 1700. Referring to FIG. 15, the guidewire 1702 can be advanced through the filter 2002 that was deployed in the branch. Furthermore, the guidewire 1702 can be advanced such that the guidewire 1702 extends out the filter's access site to create a through-and-through configuration of the guidewire 1702. FIG. 16 illustrated each of the branches (e.g., brachiocephalic, left common carotid, and left subclavian arteries 24, 25, 26) being cannulated with corresponding articulatable secondary guidewires 1700, where the guidewires 1700 extend through a corresponding side branch portal 110.

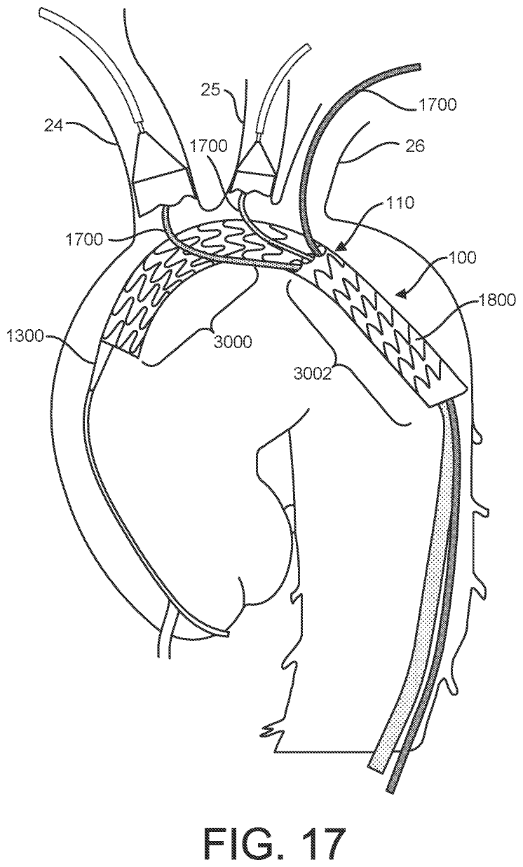

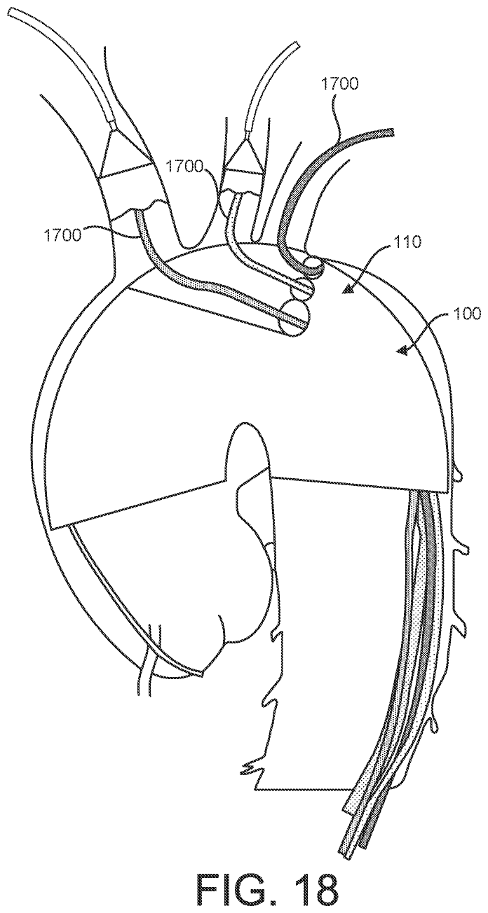

Referring now to FIG. 17, the first region 3000 is partially deployed to the second, partially constrained diameter. When the first region 3000 is partially deployed, the entire main body 100 is partially deployed to the second, partially constrained diameter. At this stage, the main body 100 can be adjusted to the appropriate position within the target site to facilitate optimal placement and performance of the implantable device 10. Once the desired positioning of the main body 100 is achieved, the main body 100 can be deployed to a third, deployed diameter (e.g., not constrained by the constraining member 1800). As illustrated in FIG. 18, once the main body 100 is fully deployed, portions of the delivery system 1000 may be removed, including the elongate member 1200 and cap 1300, the at least one guide member 1400, the at least one sheath 1600, and the constraining members 1800. As previously described, the guide members 1400 may be released, which may occur at this point in the procedure. In some embodiments, the main guidewire 1500 may also be removed. This results in the main body 100 remaining at the target site with secondary, articulatable guidewires 1700 cannulating corresponding side branch portals 110 and branches (e.g., brachiocephalic, left common carotid, and left subclavian arteries 24, 25, 26).

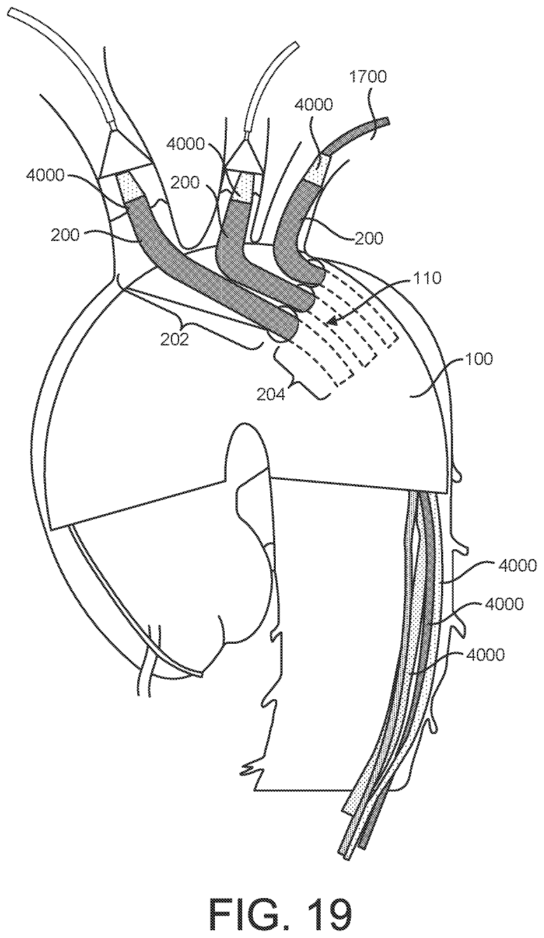

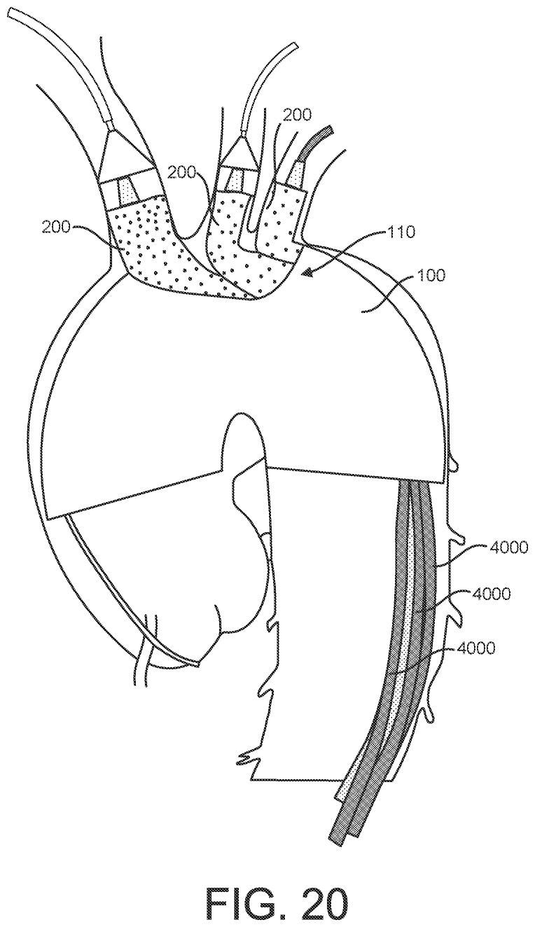

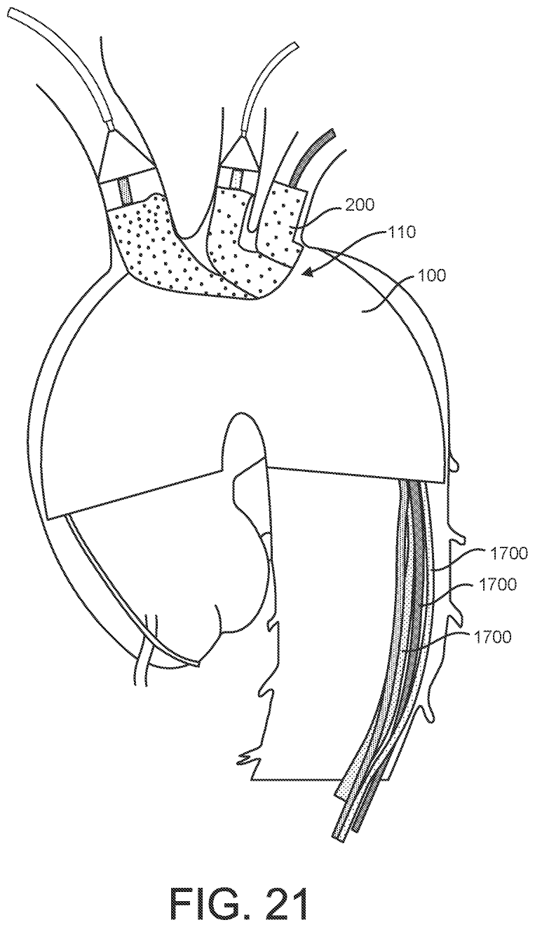

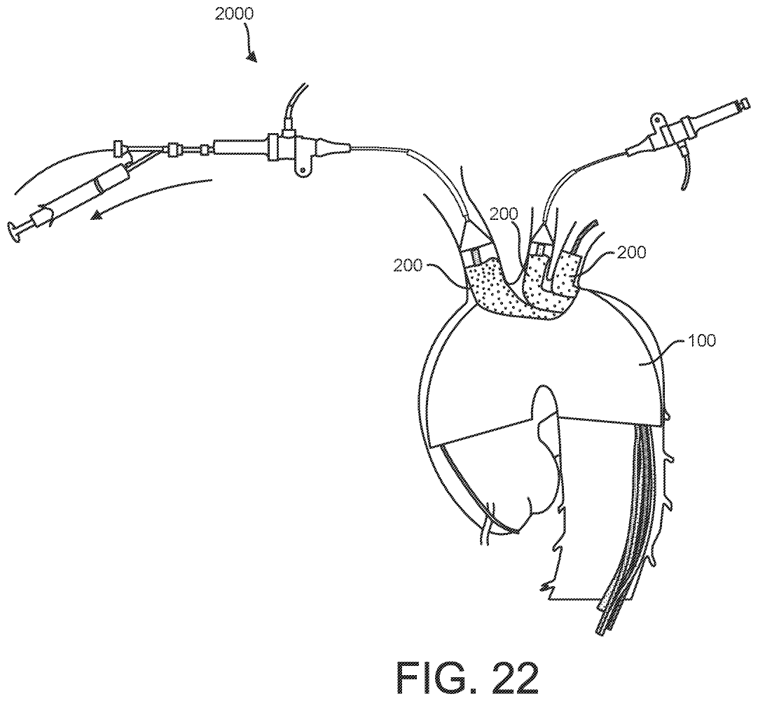

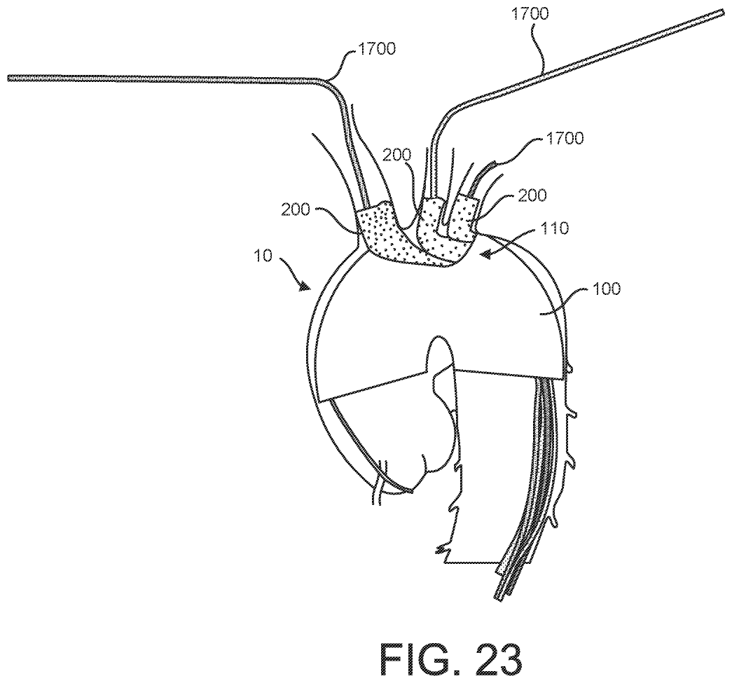

Referring to FIG. 19, branch bodies 200 can be advanced along corresponding secondary, articulatable guidewires 1700. The branch bodies 200 can be advanced on independent components, for example, elongate members 4000 with end caps 4002, and so forth, similar to the components used to deliver the main body 100. The end caps 4002, or other independent components, can dilate the side branch portals 110 in some embodiments as the branch bodies 200 are passing through the side branch portals 110. The branch bodies 200 are positioned such that a first portion 202 is at least partially positioned in the branch of the target site and a second portion is positioned within the implantable device 10 (e.g., within the side branch portal 110). Once the branch bodies 200 are appropriately positioned, the branch bodies 200 are deployed, as illustrated in FIG. 20. Referring to FIG. 21, the elongate members 4000 and end caps 4002 used to deliver the branch bodies 200 are removed. FIG. 22 illustrates aspiration of the filtration system 2000. Once, the filtration system 2000 is aspirated, the filtration system 2000 can be removed, as illustrated in FIG. 23. The remaining components can be removed from the patient (e.g., the main guidewire 1500 and the secondary, articulatable guidewires 1700) and the surgeon can commence closure.

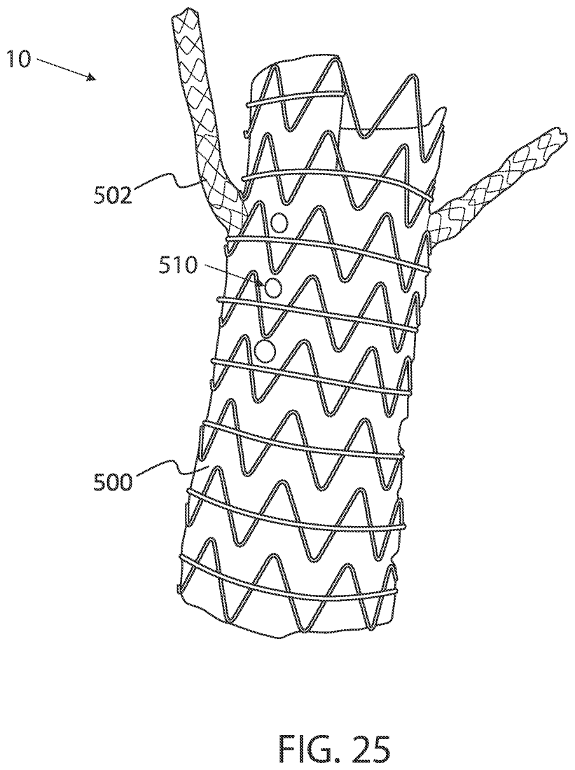

Referring to FIGS. 25-28, in some embodiments, another embodiment of the device 10 is provided with a plurality of selectable side branch portals 510. FIG. 25 illustrates a side view of an example of an implantable device 10 having a main body 500 and a plurality of selectable side branch portals 510 extending therethrough. The implantable device 10 also includes the side branches 502 extending from the main body 500 through the selectable side branch portal 510. The side branches 502 are separate from the main body 500 (i.e., they are not integral with the main body 500). Because the side branches 502 are separate structures from the main body 500, the side branches 502 are coupled to the main body 500 to form the implantable device. For example, the main body 500 may be deployed in the abdominal aorta and the side branches 502 may be deployed in the renal arteries and extend into the main body 500 positioned in the abdominal aorta.

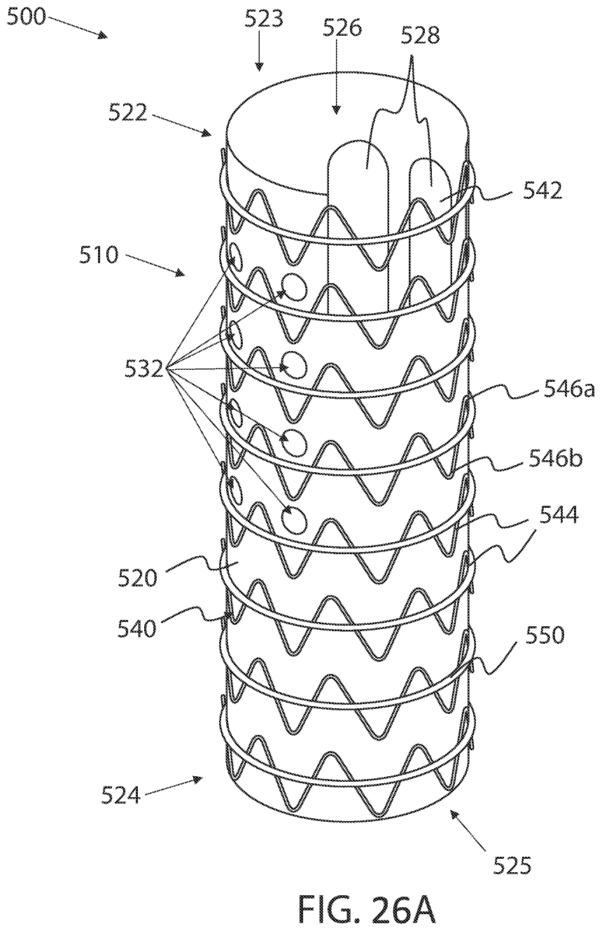

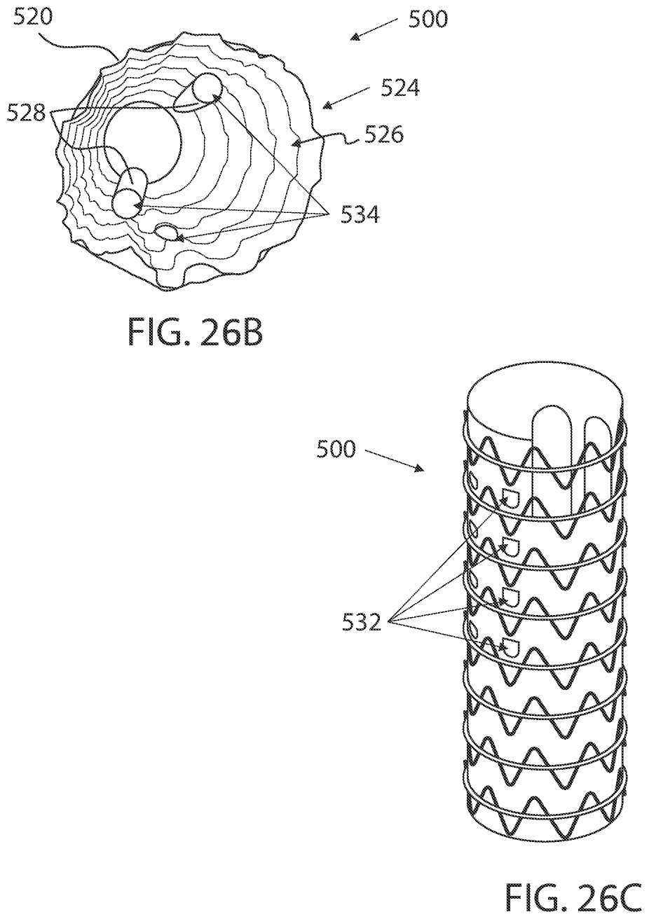

As shown in FIG. 26A, in some embodiments the main body 500 of the implantable device 10 includes a tubular member 520 and a stent member 540. As shown, the tubular member 520 has a first end 522 and a second end 524. The tubular member 520 forms a primary lumen 526 having a first opening 523 at the first end 522 and a second opening 525 at the second end 524 of the tubular member 520. The tubular member 520 includes a side branch portal 510 that includes a column 528 positioned within the primary lumen 526 and forms a secondary lumen 530 (see FIG. 26B). The tubular member 520 defines an aperture 532 into the secondary lumen 530 at a position longitudinally between the first end 522 and second end 524 of the tubular member 520. The column 528 defines a column opening 534 (see FIG. 26B) proximal the second end 524 of the tubular member 520. The stent member 540 supports the tubular member 520 in such a manner that the implantable device is operable to be configured in a delivery configuration and in a deployed configuration, or to be transitioned from a delivery configuration toward a deployed configuration.

In some embodiments, the tubular member 520 includes a first graft member 541 defining the primary lumen 526 and a second graft member 542 coupled to the first graft member 541 to form the column 528 defining the secondary lumen 530 between the first graft member 541 and the second graft member 542. For example, the first graft member 541 includes graft material formed in the shape of a tube to define the primary lumen 526. The second graft member 542 optionally includes graft material that is coupled to the first graft member 541 (e.g., via boding, adhesive, or by otherwise being coupled together) to form the secondary lumen 530. The graft materials of the first and second graft members 541, 542 may be the same material or different materials as desired. Though some materials may provide certain advantages over others, a variety of suitable graft materials may be implemented, and generally any suitable graft material may be implemented including those materials discussed herein.

In some embodiments the secondary lumen 530 extends at least partially along a longitudinal length of the main body 512. The secondary lumen 530 of the column 528 opens into the primary lumen 526 at the proximal opening of the secondary lumen 530. In some embodiments, the column 528 extends to the second end 524 of the tubular member 520 such that the column opening 534 is positioned at or coplanar with the second opening 525 of the tubular member 520. In other embodiments, the column 528 extends toward the second end 524 of the tubular member 520 such that the column opening 534 is longitudinally spaced from the second opening 525 of the tubular member 520. In embodiments including a plurality of columns 528, the column openings 534 may be positioned at the same longitudinal length across, or in different terms, at the same longitudinal position along, the tubular member 520 or they may be staggered at two or more longitudinally-spaced positions along the length of the tubular member 520.

In some embodiments, the column 528, and consequently the secondary lumen 530 are collapsible. For example, the column 528 may be unsupported by a stent member, although supported, collapsible embodiments are also contemplated. Lack of a support, or a suitably configured support, may allow the column 528 to be collapsed (radially collapsed) to seal the aperture 532 and limit the leaking or other passing of fluids (e.g., blood) through the aperture 532. In some embodiments, the pressure (e.g., hydrostatic pressure, fluid pressure gradients, and/or pressure exerted by fluids in motion) that is exerted by the fluid collapses the column 528 such that the column coapts or seals against the tubular member 520 to limit flow through the secondary lumen 530 and consequently the aperture 532.

As illustrated in FIG. 26A, the column 528 may be sealed or closed near the first end 522 of the tubular member 520, or, in some embodiments not shown, at the first end 522. The secondary lumen 530 thus is operable to provide fluid communication between the exterior surface of the tubular member 520 between the first and second end 522, 524 and the primary lumen 526, for example, when the column 528 is patent. In some embodiments, the tubular member 520 may include a column 528 that is unsealed (i.e., includes an opening) near the first end of the tubular member 520. In such embodiments, an elongate member such as a delivery catheter may be positioned through the column 528. Referring to FIG. 26B, an end view of the main body 512 is shown with the column opening 534 positioned proximate the second end 524 of the main body 512. In some embodiments, columns 528 extend to the second end 524 of the main body 512. As illustrated, the secondary lumen 530 may be contained within the primary lumen 526.

Referring again to FIG. 26A, the main body 512 includes the stent member 540. The stent member 540 may be formed of any suitable material as is discussed hereafter. The stent member 540 is operable to support the tubular member 520. The stent member 540 may be compressed into a delivery configuration and may be expanded into an expanded configuration, such as at deployment. The stent member 540 may be a self-expanding stent or a balloon expandable stent. As illustrated, the stent member 540 includes a plurality of stent rings 544. Each stent ring 544 circumferentially supports the tubular member 520 at a longitudinal position along the length of the tubular member 520. For example, each stent ring 544 is longitudinally spaced from an adjacent stent ring 544. The stent rings 544 may each include apices 546 with first apices 546a pointing toward the first end 522 and second apices 546b pointing toward the second end 524. Various other configurations of stent members 40 are contemplated herein including, but not limited to, helical stents (including undulating helical stents, diamond pattern stents, and others).

As illustrated in FIG. 26A, the tubular member 520 includes a plurality of apertures 532 spaced along the longitudinal length of the main body 512. The apertures 532 may be positioned such that at least one stent ring is between the two longitudinally adjacent apertures 532. For example, a column 528 may include apertures 532 through the tubular member 520 such that the apertures 532 are longitudinally spaced along the main body 512. The apertures are all in fluid communication with the secondary lumen 530 of the column 528. The apertures 532 provide access points for the secondary branch at various longitudinal lengths along the main body 512.

Referring to FIG. 26C, the apertures 532 may be formed in a variety of shapes and size including circular profiles, a profile with a rounded edge and a substantially flat edge, ovular profiles, and so forth. The various shapes and sizes may be implemented to accommodate various side branches 502 and configurations such as angle of exit of the side branches 502 from the main body 512 at the apertures 532. In some embodiments not shown, the apertures 532 may be irregularly spaced along the longitudinal length of the column 528. Furthermore, in some embodiments not shown, the apertures 532 may be circumferentially spaced within a column 528. For example, the apertures 532 may be staggered circumferentially and/or longitudinally.

In some embodiments, the main body 512 may include a plurality of columns 528. For instance, the main body 512 may include two columns 528 that are circumferentially spaced from each other in order to deploy two side branches 514 into the side branch lumens of the patient's anatomy. Furthermore, the main body 512 may include a plurality of columns 528 that are associated with each side branch lumen of the patient's anatomy. For example, if the main body 512 is to be deployed in the abdominal aorta and the side branches 514 are to be deployed into the renal arteries, each patient may have a various positions circumferentially at which the renal arteries enter the aorta.

By having a plurality of columns 528 through which each side branch 514 may be deployed, the surgeon may select the appropriate columns 528 that best conform to the patient's native anatomy without applying torsion to the vessels when the implantable device 10 is deployed. Thus, in one example, the main body 512 may include three columns 528 on one circumferential side of the tubular member 520 and three more columns 528 on an opposite circumferential side of the tubular member 520. Each column 528 is circumferentially spaced from the adjacent column 528 about the circumference of the tubular member 520. It is contemplated that any number of columns 528 and the spacing of the columns 528 may be implemented, including one, two, three, four, five, six, seven, eight, or more columns 528 which may be spaced equally or variably about the circumference of the tubular member 520. It is further contemplated that the specific spacing may be determined by surveying the average circumferential spacing of side branches for a particular implementation in a sample population of patients to determine the spacing of the columns 528. Circumferential spacing of the column 528 allows for clocking of the main body 512 within the patient's anatomy with increased positions for appropriately positioning the side branches 514 into the side branch vessels. As used herein, the term “clocking” refers to the ability to position features at a desired location about a circumference of an object. This ability to clock the one or more columns 528 can be further advantageous for use with visualization, for example when the procedure is being performed via fluoroscopy. This simplifies placement by providing several entry points when dealing with the two-dimensional planes shown by visualization techniques and for parallax associated with such visualization. In some embodiments, the columns 528 may be irregularly spaced about the circumference of the main body 512 (e.g., non-uniform spacing between the columns 528). In some embodiments not shown, the column 528 extends longitudinally and at angle greater than zero relative to the main body 512 longitudinal axis. For example, the secondary lumen 530 extends along a secondary lumen axis that extends longitudinally at an angle greater than zero relative to an axis of the primary lumen 526 (e.g., helically about the main body 512).

Referring again to FIG. 26A, the main body 512 may include a constraining member receiver 50 positioned surrounding at least a portion of the stent member 40. For example, in those embodiments including a plurality of stent rings 544, a corresponding constraining member receiver 550 is positioned about each stent ring 544. The constraining member receiver 550 may be formed from a variety of materials including graft materials, fibers, and so forth. The constraining member receiver 550 is operable to receive constraining members that can be retracted to partially constrain or collapse the stent rings 544 as is discussed hereafter.

In some embodiments, the tubular member 520 may include a scallop 552 at the first end 522. The scallop 552 is a facilitates placement of the tubular member 520 in a lumen including a side branch lumen that does not need a prosthetic side branch deployed. For example, when the implantable device 10 is positioned in the abdominal aorta and the superior mesenteric artery does not need a side branch 514 deployed therein, the scallop 552 may be positioned over the entrance into the superior mesenteric artery without blocking or restricting blood perfusion therethrough. The scallop 552 may include various shapes including straight edge profiles, curved profiles, and combinations thereof.

Referring now to FIGS. 27A-27C, a catheter olive or cap 1300 is positioned at the first end of the elongate member 1200 such that the main body 512 of the implantable device 10 is positioned longitudinally between the cap 1300 and the second end of the elongate member 1200. Although an embodiment of the cap 1300 is depicted in the drawings, it is in the scope of the disclosure that any catheter olive or cap may be implemented within the scope of this disclosure. The cap 1300 may be implement to atraumatically advance the delivery system 1000 through the patient and to dilate the surrounding anatomy where appropriate. For example, the cap 1300 may include a leading tip which is advanced first through the patient's anatomy. Referring to FIGS. 27A-27C, the cap 1300 may include a guide member retainer 1302. However, the guide member retainer 1302 may comprise a passage through which the guide members 1400 pass (see FIG. 27A). In this embodiment, the guide members 1400 may pass through the cap 1300 and extend back through an aperture 532 of another, oppositely positioned column 528. The guide member retainer 1302 may be operable to releasably retain a lock wire 1900 to which the guide members 1400 may be coupled (see FIG. 27B). The lock wire 1900 may be controlled via the lock wire lumen. The guide member retainer may be operable to received and releasably retain ends of the guide members 1400, for example via a friction fit or other coupling (see FIG. 27C). Various embodiments of caps 1300 may be implemented specifically for coupling the guide members 1400 (e.g., the guide member retainers 1302). Such embodiments include those discussed in U.S. Pat. Pub. No. 2020/0046534 by Chung et al., filed Aug. 13, 2019, the content of which is hereby expressly incorporated by reference. In some embodiments, the cap 1300 may be curved to facilitate clocking of the device 10 as it is advanced to the target site from implantation.

Although the method was disclosed with reference to the aorta 20, the systems and method described herein could be implemented on various lumens where branching occurs.

Catheters, introducer sheaths, hubs, handles and other components usable in medical device delivery systems and methods disclosed herein can be constructed using any suitable medical grade material or combination of materials using any suitable manufacturing process or tooling. Suitable medical grade materials can include, for example, nylon, polyacrylamide, polycarbonate, polyethylene, polyformaldehyde, polymethylmethacrylate, polypropylene, polytetrafluoroethylene, expanded polytetrafluoroethylene, polytrifluorochlorethylene, polyvinylchloride, polyurethane, elastomeric organosilicon polymers, Pebax® polyether block amide, and metals such as stainless steels and nitinol. Catheters can also include a reinforcing member, such as a layer of metal braid.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

It is understood that any of the components of the systems can also include radiopaque markers to facilitate viewing on an x-ray fluoroscope during an implantation procedure. Any number, shape and location of radiopaque markers can be utilized as needed.

Delivery systems and methods disclosed herein are particularly suited for endoluminal delivery of branchable expandable implants for treating branched vasculature. Expandable implants can include, for example, stents, grafts, and stent grafts. Further, expandable implants can include one or more stent components with one or more graft members disposed over and/or under the stent, which can dilate from a delivery configuration, through a range of larger intermediary configurations, and toward a deployed configuration engaged with vessel walls at a treatment site. However, and as discussed below, any suitable combination and configuration of stent component(s) and graft member(s) is within the scope of the present disclosure. For example, stent components can have various configurations such as, for example, rings, cut tubes, wound wires (or ribbons) or flat patterned sheets rolled into a tubular form. Stent components can be formed from metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol and biologically derived materials such as bovine arteries/veins, pericardium and collagen. Stent components can also comprise bioresorbable materials such as poly(amino acids), poly(anhydrides), poly(caprolactones), poly(lactic/glycolic acid) polymers, poly(hydroxybutyrates) and poly(orthoesters).

Moreover, potential materials for graft members include, for example, expanded polytetrafluoroethylene (ePTFE), polyester, polyurethane, fluoropolymers, such as perfluoroelastomers and the like, polytetrafluoroethylene, silicones, urethanes, ultra-high molecular weight polyethylene, aramid fibers, and combinations thereof. Other embodiments for a graft member material can include high strength polymer fibers such as ultra-high molecular weight polyethylene fibers (e.g., Spectra®, Dyneema Purity®, etc.) or aramid fibers (e.g., Technora®, etc.). The graft member may include a bioactive agent. In one embodiment, an ePTFE graft includes a carbon component along a blood contacting surface thereof. Any graft member which can be delivered by a catheter is in accordance with the present disclosure.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system. Other materials may also be used as appropriate, including but not limited to NiTiCo.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

Any of a variety of bio-active agents may be implemented with any of the foregoing. For example, any one or more of (including portions thereof) the implantable device 10 and the delivery system 1000 may comprise a bio-active agent. Bio-active agents can be coated onto one or more of the foregoing features for controlled release of the agents. Such bio-active agents can include, but are not limited to, thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, aminoglutethimide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolate mofetil); angiogenic agents (e.g., vascular endothelial growth factor (VEGF)), fibroblast growth factor (FGF); angiotensin receptor blockers; nitric oxide donors; anti-sense oligonucleotides and combinations thereof; cell cycle inhibitors, mTOR inhibitors, growth factor receptor signal transduction kinase inhibitors; retinoids; cyclin/CDK inhibitors; HMG co-enzyme reductase inhibitors (statins); and protease inhibitors. Delivery systems and methods in accordance with various embodiments disclosed herein can utilize removable guidewires to preserve branch portals for guidewire cannulation therethrough subsequent to compacting the expandable implant toward a delivery configuration for endoluminal delivery to the treatment site. Removable guidewire tube can comprise the same materials listed above for the catheter materials.

Numerous characteristics and advantages of the present invention have been set forth in the preceding description, including preferred and alternate embodiments together with details of the structure and function of the invention. The disclosure is intended as illustrative only and as such is not intended to be exhaustive. It will be evident to those skilled in the art that various modifications may be made, especially in matters of structure, materials, elements, components, shape, size and arrangement of parts within the principals of the invention, to the full extent indicated by the broad, general meaning of the terms in which the appended claims are expressed. To the extent that these various modifications do not depart from the spirit and scope of the appended claims, they are intended to be encompassed therein. In addition to being directed to the embodiments described above and claimed below, the present invention is further directed to embodiments having different combinations of the features described above and claimed below.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A method of deploying a multibranch stent graft at a target site having a main lumen and a first branch lumen, the method comprising:
advancing a main guidewire to a target site;
advancing a catheter including a main body of a multibranch stent graft along the main guidewire toward the main lumen of the target site, the main body having a first portion and a second portion, the main body defining a first portal operable to provide fluidic access from the main body to a first side branch extending from the target site when the main body is deployed at the target site, the first portal being pre-cannulated with a first secondary guidewire prior to advancing the main body along the main guidewire;
partially deploying the second portion of the main body in the main lumen of the target site;
advancing a first sheath along the first guide member through the first portal;
advancing a first articulatable guide catheter through the first sheath;
positioning the first articulatable guide catheter into a first branch lumen of the target site;
partially deploying the first portion of the main body in the main lumen of the target site;
fully deploying the first portion and the second portion of the main body;
advancing a first side branch body along the first articulatable guide catheter into the first branch lumen of the target site; and
deploying the first side branch body in the first branch lumen of the target site.

2. The method of claim 1, further comprising deploying an embolic filter in the first branch lumen of the target site.

3. The method of claim 2, further comprising aspirating a filter sheath of the embolic filter.

4. The method of claim 3, further comprising removing the embolic filter after the first side branch body has been deployed.

5. The method of claim 1, wherein the first guide member is retained proximate to a first end of the elongate member.

6. The method of claim 1, wherein the main body further defines a second portal and a third portal operable to provide fluidic access from the main body to a second side branch and a third side branch extending from the target site when the main body is deployed at the target site, the second portal being pre-cannulated with a second guide member and the third portal being pre-cannulated with a third guide member prior to advancing the main body along the main guidewire.

7. The method of claim 6, further comprising:
advancing a second sheath along the second guide member through the second portal;
advancing a second articulatable guide catheter through the second sheath;
positioning the second articulatable guide catheter into a second branch lumen of the target site;
advancing a third sheath along the third guide member through the third portal;
advancing a third articulatable guide catheter through the third sheath; and
positioning the third articulatable guide catheter into a third branch lumen of the target site.

8. The method of claim 7, further comprising:
advancing a second side branch body along the second articulatable guide catheter into the second branch lumen of the target site;
deploying the second side branch body in the second branch lumen of the target site;
advancing a third side branch body along the third articulatable guide catheter into the third branch lumen of the target site; and
deploying the third side branch body in the third branch lumen of the target site.

9. The method of claim 8, further comprising removing the main guidewire, the first, second, and third guide members, and the first, second, and third sheaths.

10. The method of claim 9, wherein the catheter is removed prior to advancing the first, second, and third sheaths.

11. An endoprosthesis delivery system comprising:
an elongate member having a first end and a second end;
an end cap coupled to the first end of the elongate member;
an endoprosthesis including a main body defining a main lumen and at least one side branch portal and at least one second body defining a secondary lumen; and
at least one guide member extending through the at least one side branch portal and coupled to the end cap.

12. The endoprosthesis delivery system of claim 11, further comprising a constraining member constraining the main body of the endoprosthesis to the elongate member.

13. The endoprosthesis delivery system of claim 12, wherein the constraining member is operable to constrain the main body at a constrained configuration and at a partially deployed configuration, the main body having a first diameter at the constrained configuration, a second diameter at the partially deployed configuration that is greater than the first diameter, and a third diameter at a deployed configuration that is greater than the first diameter and the second diameter.

14. The endoprosthesis delivery system of claim 13, wherein the constraining member includes a first portion and a second portion, wherein the first portion and the second portion are operable to independently constrain corresponding first and second portions of the main body at the constrained configuration and the partially deployed configuration.

15. The endoprosthesis delivery system of claim 11, further comprising a sheath operable to be advanced along the at least one guide member.

16. The endoprosthesis delivery system of claim 15, further comprising an articulatable wire or guide catheter operable to be advanced through the sheath.

17. The endoprosthesis delivery system of claim 16, further comprising at least one secondary branch operable to be advanced along the articulatable wire or guide catheter and to be deployed at least partially within the at least one side branch portal.

18. The endoprosthesis delivery system of claim 17, further comprising a removeable filter operable to be deployed downstream from a target site of the endoprosthesis.

19. The endoprosthesis delivery system of claim 18, wherein the removeable filter includes a central lumen through which the articulatable wire or guide catheter is operable to extend.

20. The endoprosthesis delivery system of claim 11, wherein the end cap is curved.

21. The endoprosthesis delivery system of claim 11, wherein the endoprosthesis delivery system is curved from the end cap through the main body.

22. An endoprosthesis delivery system comprising:
an elongate member having a first end and a second end;
an endoprosthesis positioned longitudinally between the first end and second end of the elongate member, the endoprosthesis including a main body defining a main lumen and a side branch portal;
a guide member extending through the side branch portal; and
a guide member retainer removably coupled to the elongate member at a coupling position, the guide member being coupled to the guide member retainer at a position between the side branch portal and the coupling position of the guide member retainer.

23. The endoprosthesis delivery system of claim 22, wherein the main body defines a plurality of side branch portals.

24. The endoprosthesis delivery system of claim 22, further comprising a plurality of guide members.

25. The endoprosthesis delivery system of claim 22, wherein the guide member retainer extends through loops formed at an end of each of the guide members.

26. The endoprosthesis delivery system of claim 22, wherein the guide member retainer is operable to be selectively decoupled from the first coupling position.

27. The endoprosthesis delivery system of claim 22, wherein the elongate member includes a lock wire retainer positioned at the first end of the elongate member.

28. The endoprosthesis delivery system of claim 27, wherein the guide member retainer is releasably coupled to the lock wire retainer.

29. The endoprosthesis delivery system of claim 22, further comprising a side branch body, wherein each guide member includes a first end, wherein each first end of the guide members is retained by the guide member retainer between the coupling position and the side branch portal when the side branch body is advanced along the guide member.

30. The endoprosthesis delivery system of claim 22, wherein each guide member is operable to be removed from a corresponding side branch portal when the guide member retainer is released.

31. The endoprosthesis delivery system of claim 22, further comprising a plurality of guide member retainers, wherein each guide member retainer is coupled to a corresponding guide member.

32. The endoprosthesis delivery system of claim 29, wherein each guide member retainer is operable to be individually and selectively released from engagement at the first coupling position such that each guide member is operable to be individually removed from a corresponding side branch portal.

33. The endoprosthesis delivery system of claim 22, wherein the elongate member includes a cap positioned at the first end of the elongate member, wherein the guide member retainer is coupled to the cap at the coupling position.

34. The endoprosthesis delivery system of claim 22, wherein the guide member retainer is coupled to the elongate member at the first end of the elongate member.
